SEQUENCE LISTING (8 sequences)

SEQ ID NO: 1 - DNA, 18 bp, Artificial Sequence
  Description of Combined DNA/RNA Molecule Synthetic oligonucleotide
  Description of Artificial Sequence Synthetic oligonucleotide
cgtgtcgccctuattccg

SEQ ID NO: 2 - DNA, 18 bp, Artificial Sequence
  Description of Artificial Sequence Synthetic oligonucleotide
cggaataagggcgaaggg

SEQ ID NO: 3 - DNA, 18 bp, Artificial Sequence
  Description of Combined DNA/RNA Molecule Synthetic oligonucleotide
  Description of Artificial Sequence Synthetic oligonucleotide
  modified_base (13): Tetrahydrofuran abasic analog
cgtgaaagactungtccg

SEQ ID NO: 4 - DNA, 18 bp, Artificial Sequence
  Description of Artificial Sequence Synthetic oligonucleotide
cggaataagtctttcacg

SEQ ID NO: 5 - DNA, 18 bp, Artificial Sequence
  Description of Artificial Sequence Synthetic oligonucleotide
  modified_base (13): 2-aminopurine
cgtgtcgcccttnttccg

SEQ ID NO: 6 - DNA, 24 bp, Artificial Sequence
  Description of Artificial Sequence Synthetic oligonucleotide
cactatcggaataagggcgaaggg

SEQ ID NO: 7 - DNA, 12 bp, Artificial Sequence
  Description of Artificial Sequence Synthetic oligonucleotide
attccgatagtg

SEQ ID NO: 8 - DNA, 40 bp, Artificial Sequence
  Description of Artificial Sequence Synthetic oligonucleotide
aacatatccgtgtcgcccttattccgatagtgactacagc

(19) United States
(12) Patent Application Publication
(10) Pub. No.: US 2008/0020973 A1
(43) Pub. Date: Jan. 24, 2008

(54) Assays for identification of topoisomerase inhibitors

(75) Inventors: James T. Stivers, Baltimore, MD (US); Keehwan Kwon, Glen Burnie, MD (US)

Correspondence Address: EDWARDS ANGELL PALMER & DODGE LLP;Client: JHU, P.O. BOX 55874, BOSTON, MA, 02205, US

(73) Assignee: The Johns Hopkins University, Baltimore, MD (US)

(21) Appl. No.: 11/701,981

(22) Filed: Feb. 1, 2007

Related U.S. Application Data
(63) Continuation of application No. PCT/US05/27605, filed on Aug. 3, 2005.
(60) Provisional application No. 60/598,395, filed on Aug. 3, 2004.
(60) Provisional application No. 60/693,252, filed on Jun. 23, 2005.

(51) Int. Cl. A61K 31/05; A61K 31/04; A61K 31/08; A61K 31/10; A61K 31/195; A61K 31/285; A61K 31/34; A61K 31/36; A61K 31/405; A61P 43/00; A61K 38/00; A61K 31/56; A61K 31/555; A61K 31/535; A61K 31/505; A61K 31/497; A61K 31/47; A61K 31/44; G01N 33/53; C12Q 1/68; A61K 31/40; A61K 31/35; A61K 31/29; A61K 31/28; A61K 31/19; A61K 31/095; A61K 31/045

(52) U.S. Cl. 514/10; 435/6; 435/7.4; 514/11; 514/177; 514/185; 514/231.8; 514/252.12; 514/274; 514/275; 514/280; 514/285; 514/292; 514/293; 514/295; 514/314; 514/336; 514/337; 514/352; 514/410; 514/414; 514/415; 514/428; 514/456; 514/464; 514/470; 514/473; 514/495; 514/503; 514/504; 514/563; 514/567; 514/570; 514/706; 514/709; 514/711; 514/730; 514/732; 514/734; 514/741

(57) ABSTRACT

The instant invention provides a continuous spectroscopic assay for DNA topoisomerase activity. The invention further provides topoisomerase inhibitors and pharmaceutical compositions for the treatment of topoisomerase associated diseases and disorders.

### RELATED APPLICATIONS

This application is a continuation of PCT/US05/27605, filed Aug. 3, 2005, which claims the benefit of U.S. Provisional Application No. 60/598,395, filed on Aug. 3, 2004, and to U.S. Provisional Application No. 60/693,252, entitled “Fluorescence Assay for Type IB DNA Topoisomerase” filed on Jun. 23, 2005, the entire contents of each of which are expressly incorporated herein by reference.

### GOVERNMENT SUPPORT

This invention was funded, at least in part, by Public Health Service Grant No.: GM-68626. Accordingly, the government may have certain rights to this invention.

### BACKGROUND OF THE INVENTION

Type IB DNA topoisomerases (topo I) catalyze the reversible cleavage of the phosphodiester backbone of DNA using a nucleophilic active site tyrosine residue (1). In the normal reaction, the DNA 5′-hydroxyl either serves as the leaving group during nucleophilic attack by the tyrosine, or as the nucleophile in the reverse reaction in which the tyrosine is expelled, and the DNA backbone is religated (FIG. 1A). As with many enzymes, the active site of topo I exhibits plasticity and allows alternative reactions to occur with remarkable catalytic promiscuity (2). These reactions include DNA strand exchange (3), attack of the phosphotyrosine intermediate by exogenous small molecule nucleophiles such as hydrogen peroxide and glycerol (4), and a ribonuclease (RNase) activity in which the tyrosine attacks the 3′ phosphodiester linkage of a single ribonucleotide in an otherwise DNA context (5). In the ribonuclease reaction, the 5′ hydroxyl of the DNA and the 2′ hydroxyl of the ribonucleotide compete for attack at the phosphotyrosine linkage, leading to either religation of DNA, or irreversible formation of a 2′, 3′ cyclic phosphodiester (FIG. 1B).

The type IB enzyme isolated from vaccinia virus has a unique and robust site-specific RNase activity that mimics the specificity of the normal DNA cleavage activity of the enzyme (5). The vaccinia topo I cleaves DNA at the 3′ phosphodiester of the consensus sequence CCCTT↓X in duplex DNA. The site-specific RNase activity is synthetically quite useful, and has been used to release isotopically labeled DNA generated by DNA polymerase catalyzed primer extension from the 3′ hydroxyl using labeled nucleotide triphosphate precursors (6).

Historically, DNA topoisomerases have been nearly impossible to investigate using standard steady-state kinetic methods. This general problem arises because the net enzymatic reaction using linear duplex DNA substrates regenerates the DNA substrate after each turnover. In contrast, multiple turnovers can be detected using supercoiled DNA because during the lifetime of the covalent phosphotyrosine complex the DNA can unwind, and subsequent religation of the DNA backbone results in a net reduction in the linking number, which can be detected by gel electrophoresis analysis (7). This supercoil relaxation assay is widely used, but is cumbersome and ill-suited for detailed mechanistic studies, or for high-throughput screening of inhibitors of these enzymes. A rapid and sensitive steady-state kinetic assay is long overdue given the importance of type IB topoisomerases as chemotherapy (8, 9) and antiviral drug targets (10).

### SUMMARY OF THE INVENTION

The instant invention is based, at least in part, on the discovery of a continuous spectroscopic assay for DNA topoisomerase activity. The inventors, for the first time, have demonstrated a multiple turnover assay for DNA topoisomerase using a DNA substrate having one or more ribonucleotide substitutions. These assays allow for high throughput screening methods to identify inhibitors of topoisomerase.

Accordingly, the instant invention provides screening methods, methods of treating topoisomerase associated diseases and disorders, compositions for the treatment of topoisomerase associated diseases and disorders, kits to screen for inhibitors of topoisomerase, pharmaceutical compositions for the treatment of topoisomerase associated diseases and disorders, and kits comprising pharmaceutical compositions for the treatment of topoisomerase associated diseases and disorders. The specific aspects and embodiments of the invention will be more thoroughly detailed below.

Accordingly, in one aspect, the instant invention provides a method for measuring the activity of a topoisomerase by contacting a topoisomerase with a duplex nucleic acid molecule that allows for multiple turnover of the topoisomerase comprising a fluorescent moiety covalently attached to one strand of the duplex nucleic acid molecule and a fluorescence quencher covalently attached to the complimentary strand of the duplex nucleic acid molecule, wherein topoisomerase activity results in measurable fluorescence from the fluorescent moiety, and measuring the fluorescence of the fluorescent moiety, thereby measuring the activity of the topoisomerase.

In a related embodiment the duplex nucleic acid molecule is a deoxyribonucleic acid molecule. In another related embodiment, the multiple turnover of the topoisomerase is due to the duplex nucleic acid molecule further comprising one or more ribonucleotides, or analogs thereof, e.g., a uridine ribonucleotide.

In a related embodiment, the fluorescence is measured spectroscopically, e.g., using a fluorometer.

In specific embodiments the topoisomerase is a type I topoisomerase, e.g., type IA or IB. In further specific embodiments, the topoisomerase is a vaccinia virus topoisomerase IB or a human topoisomerase IB. In another embodiment, the topoisomerase is a type II topoisomerase.

In another embodiment, the duplex nucleic acid molecule is comprised of SEQ ID NO:1 and SEQ ID NO:2. In a related embodiment, SEQ ID NO:1 contains a fluorescent moiety, e.g., FAM, and SEQ ID NO:2 contains a fluorescence quencher, e.g., DAB. In another related embodiment, the duplex nucleic acid molecule is a comprised of SEQ ID NO:1 and SEQ ID NO:2 and the topoisomerase is vaccinia virus topoisomerase IB.

In another embodiment, the duplex nucleic acid molecule is comprised of SEQ ID NO:3 and SEQ ID NO:4. In a related embodiment, the duplex nucleic acid molecule is a comprised of SEQ ID NO:3 and SEQ ID NO:4 and the topoisomerase is human topoisomerase IB.

In another embodiment, the topoisomerase is a pathogen topoisomerase, e.g., a malaria or trypanosome topoisomerase.

In another embodiment, the method further involves contacting the topoisomerase with a candidate topoisomerase inhibitor. In yet a further embodiment, the method further comprises comparing the level of activity of the topoisomerase in the absence of the candidate inhibitor to the activity in the presence of the candidate inhibitor, wherein a lower level of activity in the presence of the candidate inhibitor is indicative that the candidate inhibitor is an inhibitor.

In other aspects the instant invention provides a method of determining if a compound is an antiviral, antimalarial, antitrypanosome or antibacterial agent by creating an admixture comprising topoisomerase, a candidate antiviral, antimalarial, antitrypanosome or antibacterial agent, and a duplex nucleic acid molecule that allows for multiple turnover of the topoisomerase comprising a fluorescent moiety covalently attached to one strand and a fluorescence quencher covalently attached to the complimentary strand, wherein topoisomerase activity allows for measurable fluorescence emission from the fluorescent moiety; measuring the fluorescence of the fluorescent moiety, thereby measuring the activity of the topoisomerase, wherein, a decrease in the activity of the topoisomerase in the presence of the candidate antiviral, antimalarial, antitrypanosome or antibacterial agent compared to the level of activity of the topoisomerase in the absence of the candidate antiviral, anticancer, antimalarial, or antibacterial agent is indicative that the candidate inhibitor is a antiviral, antimalarial, antitrypanosome or antibacterial agent, respectively.

In a related embodiment the duplex nucleic acid molecule is a deoxyribonucleic acid molecule. In another related embodiment, the multiple turnover of the topoisomerase is due to the duplex nucleic acid molecule further comprising one or more ribonucleotides, or analogs thereof, e.g., a uridine ribonucleotide.

In a related embodiment, the fluorescence is measured spectroscopically, e.g., using a fluorometer.

In specific embodiments the topoisomerase is a type I topoisomerase, e.g., type IA or IB. In further specific embodiments, the topoisomerase is a viral, bacterial, malarial, or trypanosome topoisomerase IB. In another embodiment, the topoisomerase is a type II topoisomerase.

In another embodiment, the duplex nucleic acid molecule is comprised of SEQ ID NO:1 and SEQ ID NO:2. In a related embodiment, the duplex nucleic acid molecule is a comprised of SEQ ID NO:1 and SEQ ID NO:2 and the topoisomerase is vaccinia virus topoisomerase IB.

In another embodiment, the invention provides a method for testing a library of candidate antiviral, antimalarial, antitrypanosome or antibacterial agents for the ability to inhibit topoisomerase activity.

In another aspect, the instant invention provides a method of determining if a compound is an anticancer agent by creating an admixture comprising topoisomerase, a candidate anticancer agent, and a duplex nucleic acid molecule that allows for multiple turnover of the topoisomerase comprising a fluorescent moiety covalently attached to one strand and a fluorescence quencher covalently attached to the complimentary strand, wherein topoisomerase activity allows for measurable fluorescence emission from the fluorescent moiety, measuring the fluorescence of the fluorescent moiety, thereby measuring the activity of the topoisomerase, wherein, a decrease in the activity of the topoisomerase in the presence of the candidate anticancer agent compared to the level of activity of the topoisomerase in the absence of the candidate anticancer agent is indicative that the candidate inhibitor is a anticancer agent.

In a related embodiment the duplex nucleic acid molecule is a deoxyribonucleic acid molecule. In another related embodiment, the multiple turnover of the topoisomerase is due to the duplex nucleic acid molecule further comprising one or more ribonucleotides, or analogs thereof, e.g., a uridine ribonucleotide.

In a related embodiment, the fluorescence is measured spectroscopically, e.g., using a fluorometer.

In another embodiment, the duplex nucleic acid molecule is comprised of SEQ ID NO:3 and SEQ ID NO:4. In a related embodiment, the duplex nucleic acid molecule is a comprised of SEQ ID NO:3 and SEQ ID NO:4 and the topoisomerase is human topoisomerase IB.

In another embodiment, the invention provides a method for testing a library of candidate anticancer agents for the ability to inhibit topoisomerase activity.

In another aspect, the invention provides a method for treating a subject having a topoisomerase associate disease or disorder by administering to the subject an effective amount of a compound of Formula 1: 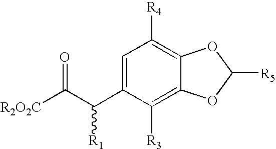
wherein: the line to R₁ indicates a single or a double bond; R₂-R₅ are each independently H, a halide, an alkyl, an aryl, a cyano, an alcohol, an amine or an amide; and R₁ is H, a halide, an alkyl, an aryl, a cyano, an alcohol, an amine or an amide;
A Compound of Formula 2: 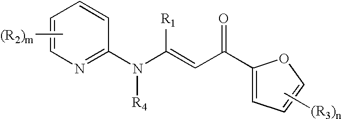
wherein: R₁ is H, a halide, an alkyl, an aryl, a cyano, an alcohol, an amine or an amide; (R₂)ₘ are each independently H, a halide, an alkyl, an aryl, a cyano, an alcohol, an amine or an amide and m=1, 2, 3 or 4; (R3)ₙ are each independently H, a halide, an alkyl, an aryl, a cyano, an alcohol, an amine or an amide and n=1, 2 or 3.
A Compound of Formula 3: 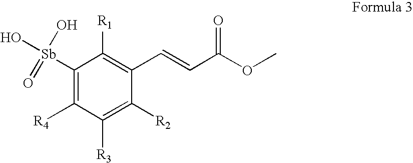
wherein: R₁-R₄ are each independently H, a halide, an alkyl, an aryl, a cyano, an alcohol, an amine or an amide;
A Compound of Formula 4: 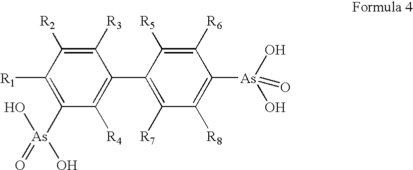
wherein: R₁-R₇ are each independently H, a halide, an alkyl, an aryl, a cyano, an alcohol, an amine or an amide;
A Compound of Formula 5: 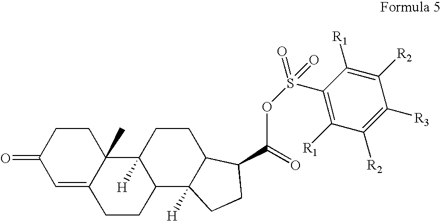
wherein: R₁-R₃ are each independently H, a halide, an alkyl, an aryl, a cyano, an alcohol, an amine or an amide;
A Compound of Formula 6: 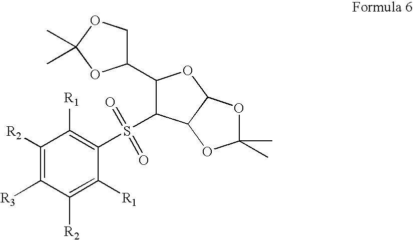
wherein: R₁-R₃ are each independently H, a halide, an alkyl, an aryl, a cyano, an alcohol, an amine or an amide;
A Compound of Formula 7: 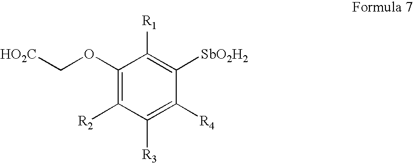
wherein: R₁, R₂, R₃, and R₄ are each independently H, an Alkyl, a halide, an aryl, a cyano, an alcohol, an amine or an amide;
A Compound of Formula 8:

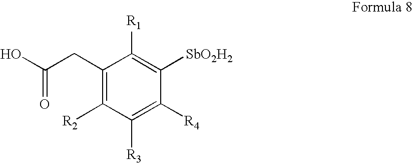

wherein: R₁, R₂, R₃, and R₄ are each independently H, an Alkyl, a halide, an aryl, a cyano, an alcohol, an amine or an amide;
A Compound of Formula 9:

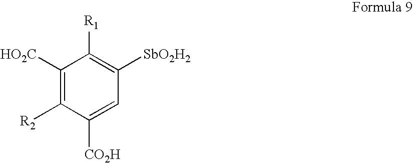

wherein: R₁, R₂, R₃, and R₄ are each independently H, an Alkyl, a halide, an aryl, a cyano, an alcohol, an amine or an amide;
A Compound of Formula 10: 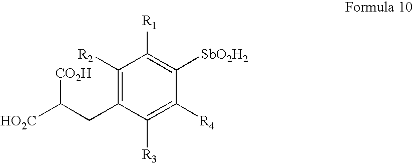
wherein: R₁, R₂, R₃, and R₄ are each independently H, an Alkyl, a halide, an aryl, a cyano, an alcohol, an amine or an amide; or
A Compound of Formula 11: 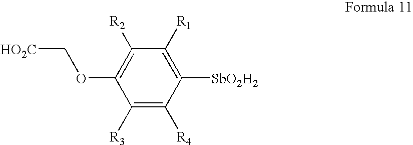
wherein: R₁, R₂, R₃, and R₄ are each independently H, an Alkyl, a halide, an aryl, a cyano, an alcohol, an amine or an amide.

In related embodiments, the topoisomerase associated disease or disorder is selected from the group consisting of cancer, viral infection or bacterial infection.

In a related aspect, the invention provides a method of treating a subject having a topoisomerase associated disease or disorder by administering to the subject an effective amount of a compound identified in Table 2, 3 4, or 5, thereby treating the subject.

In a specific embodiment, the compound is selected from the compounds listed in Table 4.

In one embodiment, the topoisomerase associated disease or disorder is selected from the group consisting of cancer, viral infection or bacterial infection.

In another aspect, the instant invention provides a pharmaceutical composition for the treatment of a topoisomerase associated disease or disorder comprising a compound identified as Formula 1, Formula 2, Formula 3, Formula 4, Formula 5, Formula 6, Formula 7, Formula 8, Formula 9, Formula 10 or Formula 11, or a compound identified in Tables 2, 3, 4, or 5, and a pharmaceutically acceptable carrier.

In one embodiment, the compounds of Formula 7, Formula 8, Formula 9, Formula 10 or Formula 11 specifically bind to human topoisomerase IB. Moreover, these compounds can be used to treat a human subject having a topoisomerase associated disease or disorder, e.g., a cell proliferative disorder such as cancer.

In a related embodiment, the pharmaceutical composition is useful in the treatment of viral infection, pathogen infection or bacterial infection.

In another aspect, the invention provides a kit comprising a pharmaceutical composition described herein and instructions for use. In a related embodiment, the kit is useful for the topoisomerase associated disease or disorder, e.g., cancer, viral infection or bacterial infection.

In another aspect, the invention provides a kit for determining if a compound is a topoisomerase inhibitor comprising, a duplex nucleic acid molecule that allows for multiple turnover of the topoisomerase comprising a fluorescent moiety covalently attached to one strand of the duplex nucleic acid molecule and a fluorescence quencher covalently attached to the complimentary strand of the duplex nucleic acid molecule, wherein topoisomerase activity results in measurable fluorescence from the fluorescent moiety, and instructions for use. In specific embodiments, the kit may further comprise a topoisomerase, e.g., a human, viral, pathogenic, or bacterial topoisomerase.

### DESCRIPTION OF THE DRAWINGS

FIG. 1 is a schematic representation of Topo I catalyzed reactions. FIG. 1A depicts the reversible site-specific cleavage and ligation reaction in which the nucleophilic tyrosine attacks a DNA phosphodiester linkage, expelling a 5′ hydroxyl leaving group. FIG. 1B depicts the irreversible ribonuclease reaction in which the enzyme attacks the 3′ phosphodiester linkage of uridine within the consensus sequence 5′-CCCTU-3′. The phosphotyrosine intermediate is then attacked by the 2′ hydroxyl group resulting in turnover of the enzyme and formation of a cyclic 2′, 3′ phosphodiester product.

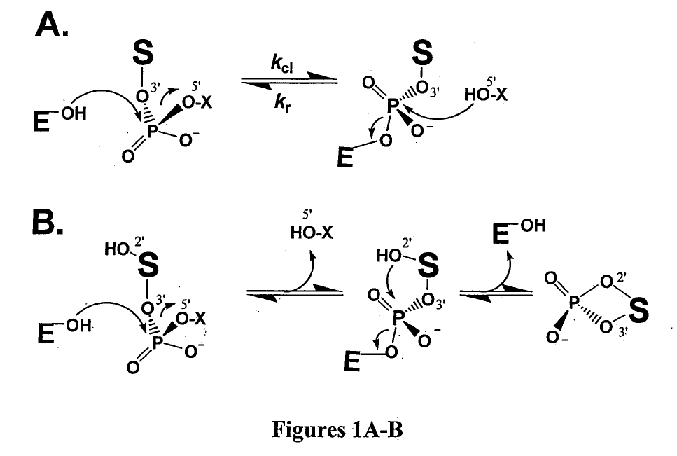

FIGS. 2A-B depict the Molecular beacon assay for topo I and fluorescence emission spectra for the free molecular beacon substrate and product of the topo I RNase reaction. FIG. 2A demonstrates that the assay relies on the strong quenching of the 3′6-carboxyfluorescein fluorophore (FAM) in the substrate by a dabcyl group (DAB) that is covalently attached to the 5′ end of the complementary strand. Upon cleavage of the scissile strand by topo I, the short strand with the FAM group is released to solution, resulting in an increase in fluorescence. Recycling of the enzyme for multiple turnovers is accomplished by attack of the 2′ hydroxyl of the uridine ribonucleotide. FIG. 2B depicts the fluorescein emission spectra for the 18U-FAM/18-DAB duplex (50 nM) before (lower spectrum) and after (upper spectrum) 30 min incubation with vaccinia DNA topoisomerase IB (vTopo). The fluorescence intensity increases by 9-fold when the reaction is completed. The final fluorescence is similar to that of the free 18U-FAM single stranded DNA (middle spectrum).

FIG. 3 depicts presteady-state kinetic analysis of the topo I RNase reaction. Ten nanomolar enzyme was incubated with the molecular beacon substrate at the following concentrations: 50 nM, 100 nM, 200 nM, 400 nM and 800 nM. The solid curves were obtained from global analysis of the time courses using computer simulation and the mechanism shown in Scheme 1. The global best-fit kinetic parameters are reported in Table 1. The kinetic constants in the individual fits that are shown were allowed to float within ±15% of the global values.

FIG. 4 depicts representative inhibitor screening data obtained using the molecular beacon assay described herein in a 96-well microtiter plate format. The figure shows the first three rows of plate 3846 of the NCI Diversity Library Collection. One of the most potent inhibitors detected in our partial screen of this library is compound 112983 (asterisk), which was subjected to further analysis. Column twelve is a negative control (substrate, omit enzyme) and column one is a positive control (substrate and enzyme, no compound).

FIGS. 5A-B depict the mechanism of inhibition of the topo I RNase activity by compound 112983. The reactions included 10 nM topo I, and either 50 nM (FIG. 5A) or 3 μM (FIG. 5B) molecular beacon substrate, and the following concentrations of 112983: 32 μM, 16 μM, 2 μM, 1 μM or 0 μM. The solid curves were obtained from global analysis of the time courses using computer simulation and the mechanism in Scheme 2. From this analysis, a Kᵢ value of 1.6 μM for binding of 112983 to the E-I complex was determined

FIGS. 6A-B depict inhibition of DNA supercoil relaxation catalyzed by vTopo and human topoisomerase type IB (hTopo). FIG. 6A depicts the results of an experiment in which vTopo and pUC19/AID supercoiled DNA were incubated with increasing concentrations of 112983 for nine minutes before quenching the reactions with 0.4% SDS. The supercoiled and relaxed DNAs were then resolved on a 1% agarose gel and detected with ethidium bromide staining. The relative intensities of the substrate and product bands were determined by fluorescence imaging to calculate the fraction of the DNA in each lane that was in the relaxed form. Lane 1: DNA alone, 3 through 11 contain 3 nM vTopo with concentrations of [112983]=0, 1, 2, 5, 10, 20 and 40 μM. Lanes 9 through 11 contain DNA and vTopo plus the following concentrations of inhibitors: Lane 9: 1000 μM novobiocin, Lane 10 and 11: 500 μM of compound 120927 or 14555, respectively (see Table 2). FIG. 6B depicts the results of an experiment in which hTopo and pUC19/AID supercoiled DNA were incubated with increasing concentrations of 112983 for 15 minutes before quenching the reactions with 0.4% SDS. The partially relaxed products were resolved by electrophoresis using a 1% agarose gel. Lane 1: DNA alone, 2 through 8 contain 2 units of hTopo with concentrations of [112983]=0, 1, 2, 5, 10, 20 and 40 μM.

FIGS. 7A-B indicate that compound 112983 does not inhibit DNA binding of Y274F vTopo. FIG. 7A depicts noncovalent binding of Y274F vTopo to FAM-18AP/24 duplex as monitored by the increase in the steady-state fluorescein anisotropy of the DNA (FAM, filled circles), or increase in 2-aminopurine fluorescence (2AP, open circles). FIG. 7B depicts steady-state anisotropy of (1) the free FAM-18AP/24 mer DNA, (2) the Y274F-FAM-18AP/24 complex, (3-5) the complex in the presence of 112983, with the order of addition of reagents as indicated, and (6) the complex and 112983 after addition of an unlabeled competitor 24/24 mer duplex (C, 40 μM) containing a CCCTT consensus sequence.

FIGS. 8A-C indicate that compound 112983 does not inhibit the single turnover DNA cleavage or ligation reactions of vTopo. FIG. 8A depicts suicide DNA cleavage reaction with fluorescence detection. vTopo was rapidly mixed with a suicide substrate DNA containing a 2-aminopurine fluorescent label in the 6 mer leaving strand (13) and the fluorescence increase was monitored using stopped-flow fluorescence. The cleavage rate was k_{cl}=2.3±0.06 s⁻¹ and k_{cl}=1.8±0.05 s⁻¹ in the absence and presence of 8 μM 112983, respectively. FIG. 8B depicts the results of a single-turnover religation reaction. A covalent complex between vTopo and a 5′-³²P-labeled 12/24 suicide DNA substrate was formed and rapidly mixed with a complementary 12 mer DNA strand that was provided in large molar excess. The 5′-OH of the 12 mer attacks the covalent phophotyrosine linkage resulting in the formation of a labeled 24 mer duplex that can be separated from the covalent complex electrophoretically using a 15% SDS-polyacrylamide gel. The ligation reaction was performed in the absence and presence of compound 112983 (32 μM). The reactions were quenched at 10 s, and the percent of the DNA that was covalently bound was determined by phosphorimaging. In the presence of 32 μM 112983, the % complex decreased from 15% to 7%, indicating that compound binding increases the religation rate. FIG. 8C depicts equilibrium DNA cleavage. vTopo (360 nM) was added to a solution of 5′-³²P-labeled 40/40 mer (300 nM) and incubated for five minutes before quenching the equilibrium cleavage-religation reaction by the addition of 5% SDS. The fraction covalent complex was determined after separating the free and covalently bound DNA by gel electrophoresis. The percent covalently bound DNA decreased by 50% in the presence of 100 μM compound (lane 3), further confirming that the compound decreases the cleavage equilibrium (K_{cl}).

### DETAILED DESCRIPTION OF THE INVENTION

The instant invention is based, at least in part, on the discovery of a continuous spectroscopic assay for DNA topoisomerase activity. The inventors, for the first time, have demonstrated a multiple turnover assay for DNA topoisomerase using a DNA substrate having one or more ribonucleotide substitutions. This assay allows for a high throughput screening assay for novel inhibitors of topoisomerase activity.

Topoisomerases have been identified as effective drug targets for the treatment of cancer and infection, e.g., viral and bacterial infection. Accordingly, the instant invention provides screening methods, methods of treating topoisomerase associated diseases and disorders, compositions for the treatment of topoisomerase associated diseases and disorders, kits to screen for inhibitors of topoisomerase, pharmaceutical compositions for the treatment of topoisomerase associated diseases and disorders; and kits comprising pharmaceutical compositions for the treatment of topoisomerase associated diseases and disorders.

Topoisomerase Assay

The instant invention provides for the first time continuous topoisomerase activity assays. These assays provided allow for monitoring of topoisomerase activity under multiple turnover conditions. In preferred embodiments of the invention, the assays are used to identify inhibitors of topoisomerase activity.

The assays require a topoisomerase, e.g., a human, viral, or bacterial topoisomerase, and a DNA substrate comprising: a duplex nucleic acid molecule that allows for multiple turnover of the topoisomerase that has a label that is not detectable when the DNA is a duplex, but is detectable when the DNA duplex is disrupted, i.e., by topoisomerase activity. In one embodiment, the assays use molecular beacon technology wherein a fluorescent label is attached to the 3′ end of one strand of the duplex and a fluorescence quencher is attached to the 5′ end of the complimentary stand of the duplex, or vice versa. Using this technology, when acted upon by topoisomerase, the fluorescent moieties released from the quencher and produces a measurable fluorescent signal.

The assays utilize a mutant DNA duplex that allows for turnover of the enzyme. In one embodiment, the duplex is:

| | |
|---|---|
| 5′ CGTGTCGCCCTUATTCCG-FAM-3′ | (SEQ ID NO:1) |
| 3′ GGGAAGCGGGAATAAGGC-DAB-5′ | (SEQ ID NO:2) |

wherein U is uridine ribonucleotide, FAM is the fluorescent label 6-carboxyfluorescein and DAB is the fluorescence quencher dabcyl. This substrate is optimized for vaccinia virus topoisomerase type I, but one of skill in the art will realize that modifications of the DNA sequence can be made to tailor the substrate to different topoisomerases, i.e., topoisomerases from different species. Accordingly, in another embodiment, the substrate is optimized for human topoisomerase IB and has the sequence:

| | |
|---|---|
| 5′ CGTGAAAGACTUφGTCCG-FAM-3′ | SEQ ID NO:3 |
| 3′ GCACTTTCTGAATAAGGC-DAB-5′ | SEQ ID NO:4 |

wherein FAM is the fluorescent label 6-carboxyfluorescein, DAB is the fluorescence quencher dabyl, U is uridine ribonucleotide and Φ is tetrahydrofuran abasic analog.

Without being bound by theory, the inventors believe that the substitution of the ribonucleotide uridine for the 3′ thymidine of the consensus cleavage sequence allows for multiple turn over of the enzyme. See the Examples.

One of skill will understand that, although the exemplary assays of the invention use fluorescent labels to measure the activity of topoisomerase, any label that can emit a different signal when attached to duplex DNA as opposed to single stranded DNA will be useful in the methods of the invention.

Accordingly, the instant invention provides methods to determine if a compound is an inhibitor of topoisomerase by contacting a topoisomerase with the DNA duplex described above and a candidate inhibitor and determining if the candidate inhibitor is capable of inhibiting the action of the topoisomerase by comparing the activity of the topoisomerase in the presence of the candidate inhibitor to the activity in the absence of the inhibitor.

In one embodiment, the activity of the topoisomerase is determined by measuring the fluorescence emission of the fluorescent tag. Using a fluorometer, one of skill in the art can measure the fluorescent emission of a fluorescent moiety by exciting the moiety with a wavelength of light that excites the particular fluorescent moiety and measuring the fluorescence emission that results. For further details see the Examples.

In specific embodiments of the invention the assays use type I topoisomerases, e.g., type IA or IB. In other embodiments, the assays use type II topoisomerases. In other specific embodiments of the invention, the topoisomerase is a human, viral, e.g., vaccinia virus, bacterial, or parasitic, e.g., from malaria or trypanosome.

The assays described herein allow for the screening of libraries of compounds for inhibition of topoisomerase. One of skill in the art can perform these assays using high throughput methods such as microtiter plates and plate reading fluorometers to rapidly screen large libraries of compounds.

In certain embodiments, the assays described herein are used to identify anticancer, antiviral, antibacterial, or antipathogenic compounds for use in treating subjects having a topoisomerase associated disease or disorder.

### COMPOUNDS OF THE INVENTION

The topoisomerase assays described herein can be used to identify compounds that are capable of inhibiting the activity of topoisomerase. Accordingly, using the assays described herein, it has been demonstrated that the compounds in Tables 2, 3, and 4 are inhibitors of topoisomerase activity.

Accordingly, based on the inhibitors identified, the instant invention provides topoisomerase inhibitors having the structure of Formula 1:

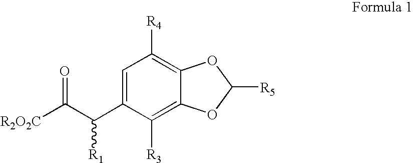

wherein: the line to R₁ indicates a single or a double bond; R₂-R₅ are each independently H, a halide, an alkyl, an aryl, a cyano, an alcohol, an amine or an amide; and R₁ is H, a halide, an alkyl, an aryl, a cyano, an alcohol, an amine or an amide.

In another embodiment, the instant invention provides topoisomerase inhibitors having the structure of Formula 2: 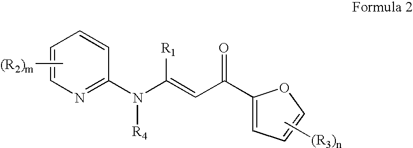
wherein: R₁ is H, a halide, an alkyl, an aryl, a cyano, an alcohol, an amine or an amide; (R₂)ₘ are each independently H, a halide, an alkyl, an aryl, a cyano, an alcohol, an amine or an amide and m=1, 2, 3 or 4; (R3)ₙ are each independently H, a halide, an alkyl, an aryl, a cyano, an alcohol, an amine or an amide and n=1, 2 or 3.

In another embodiment, the instant invention provides topoisomerase inhibitors having the structure of Formula 3: 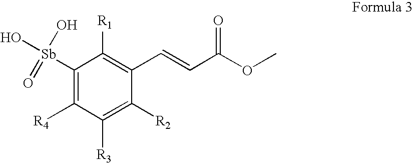
wherein: R₁, R₂, R₃, and R₄ are each independently H, a halide, an alkyl, an aryl, a cyano, an alcohol, an amine or an amide.

In another embodiment, the instant invention provides topoisomerase inhibitors having the structure of Formula 4: 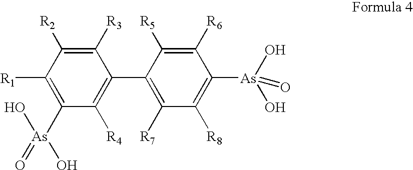
wherein: R₁, R₂, R₃, R₄, R₅, and R₆ are each independently H, a halide, an alkyl, an aryl, a cyano, an alcohol, an amine or an amide.

In another embodiment, the instant invention provides topoisomerase inhibitors having the structure of Formula 5: 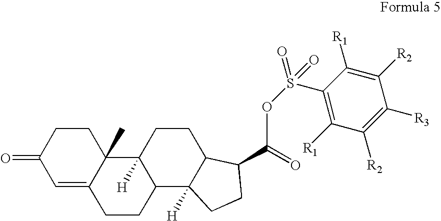
wherein: R₁, R₂ R₃, R₄, R₅, and R₆ are each independently H, a halide, an alkyl, an aryl, a cyano, an alcohol, an amine or an amide.

In another embodiment, the instant invention provides topoisomerase inhibitors having the structure of Formula 6: 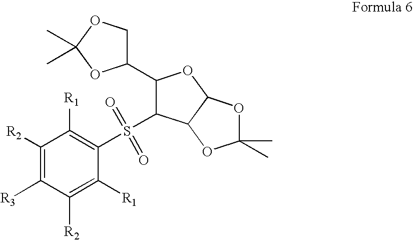
wherein: R₁, R₂, R₃, R₄, R₅, and R₆ are each independently H, a halide, an alkyl, an aryl, a cyano, an alcohol, an amine or an amide.

In another embodiment, the instant invention provides topoisomerase inhibitors having the structure of Formula 7: 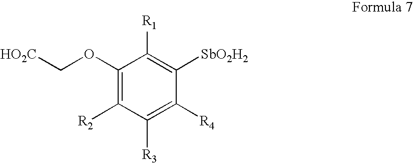
wherein: R₁, R₂, R₃, and R₄ are each independently H, an Alkyl, a halide, an aryl, a cyano, an alcohol, an amine or an amide.

In another embodiment, the instant invention provides topoisomerase inhibitors having the structure of Formula 8: 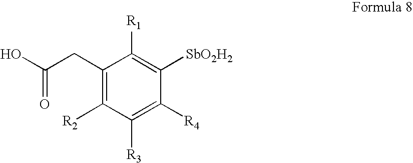
wherein: R₁, R₂, R₃, and R₄ are each independently H, an Alkyl, a halide, an aryl, a cyano, an alcohol, an amine or an amide.

In another embodiment, the instant invention provides topoisomerase inhibitors having the structure of Formula 9: 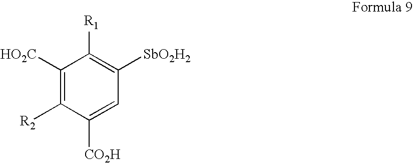
wherein: R₁, R₂, R₃, and R₄ are each independently H, an Alkyl, a halide, an aryl, a cyano, an alcohol, an amine or an amide.

In another embodiment, the instant invention provides topoisomerase inhibitors having the structure of Formula 10: 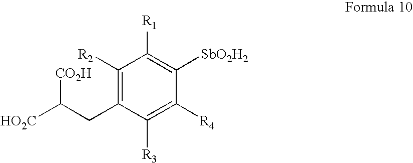
wherein: R₁, R₂, R₃, and R₄ are each independently H, an Alkyl, a halide, an aryl, a cyano, an alcohol, an amine or an amide.

In another embodiment, the instant invention provides topoisomerase inhibitors having the structure of Formula 11: 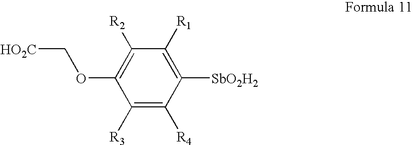
wherein: R₁, R₂, R₃, and R₄ are each independently H, an Alkyl, a halide, an aryl, a cyano, an alcohol, an amine or an amide.

In another embodiment, the topoisomerase inhibitors of Formula 7-11 are inhibitors of human topoisomerase IB.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer. Likewise, preferred cycloalkyls have from 4-7 carbon atoms in their ring structure, and more preferably have 5 or 6 carbons in the ring structure.

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)). The term “alkyl” also includes the side chains of natural and unnatural amino acids. Examples of halogenated alkyl groups include fluoromethyl, difluoromethyl, trifluoromethyl, chloromethyl, dichloromethyl, trichloromethyl, perfluoromethyl, perchloromethyl, perfluoroethyl, perchloroethyl, etc.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “amine” or “amino” includes compounds where a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group that is bound to at least one alkyl group and at least one aryl group. The term “alkaminoalkyl” refers to an alkyl, alkenyl, or alkynyl group bound to a nitrogen atom that is also bound to an alkyl group.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl, or alkynyl groups bound to an amino group bound to a carboxy group. It includes arylaminocarboxy groups that include aryl or heteroaryl moieties bound to an amino group which is bound to the carbon of a carbonyl or thiocarbonyl group. The terms “alkylaminocarboxy,” “alkenylaminocarboxy,” “alkynylaminocarboxy,” and “arylaminocarboxy” include moieties wherein alkyl, alkenyl, alkynyl and aryl moieties, respectively, are bound to a nitrogen atom which is in turn bound to the carbon of a carbonyl group.

The term “halide” includes compounds comprising a halogen with a more electropositive element or group. Exemplary halides include NaCl, KI, LiCl, CuCl₂, ClF, CH₃Br, and CHI₃.

The term “cyano” includes compounds having an monovalent CN.

The term “alcohol” includes organic compound in which a hydroxyl group is bound to a carbon atom. Alcohols can be primary, secondary, or tertiary in structure.

The compounds identified in Tables 2, 3, 4, and as Formula 1, Formula 2, Formula 3, Formula 4, Formula 5, and Formula 6 are useful in the treatment of topoisomerase associated diseases and disorders. Accordingly, the instant invention provides compounds having the structure identified as Formula 1, Formula 2, Formula 3, Formula 4, Formula 5, or Formula 6 that are inhibitors of topoisomerase activity.

Pharmaceutical Compositions

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, .alpha.-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats an topoisomerase associated disease or disorder.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition. Moreover, the pharmaceutical compositions described herein may be administered with one or more other active ingredients that would aid in treating a subject having a topoisomerase associated disease or disorder. In a related embodiment, the pharmaceutical compositions of the invention may be formulated to contain one or more additional active ingredients that would aid in treating a subject having a topoisomerase associated disease or disorder, e.g., anticancer compounds or antimalarial compounds.

Kits

The pharmaceutical compositions can be included in a container, pack, kit or dispenser together with instructions, e.g., written instructions, for administration, particularly such instructions for use of the active agent to treat against a topoisomerase associated disease or disorder. The container, pack, kit or dispenser may also contain, for example, one or more additional active ingredients that would aid in treating a subject having a topoisomerase associated disease or disorder, e.g., anticancer compounds or antimalarial compounds.

In another embodiment, the instant invention provides kits containing reagents for testing a compound for the ability to inhibit topoisomerase activity along with instructions, e.g., written instructions, for using the kit. For example, the kit may contain a duplex DNA substrate such as those described herein and instructions for use. Further, the kit may contain one or more topoisomerases to be screened against one or more compounds.

Methods of Treatment

The term “topoisomerase associated diseases and disorders” is intended to include diseases and disorders characterized by aberrant expression or activity, or a disease or disorder that relies on the activity of a topoisomerase to progress. Exemplary topoisomerase associated diseases and disorders are cancer and infection, e.g., bacterial and viral infection. Viral infections such as vaccinia family viral infections can be treated with topoisomerase inhibitors. Further, pathogenic infections such as malaria and trypanosome infection can be treated with topoisomerase inhibitors.

The term “treated,” “treating” or “treatment” includes the diminishment or alleviation of at least one symptom associated or caused by a topoisomerase associated disease or disorder. For example, treatment can be diminishment of one or several symptoms of a disorder or complete eradication of a disorder.

The term “subject” is intended to include organisms, e.g., prokaryotes and eukaryotes, which are capable of suffering from or afflicted with a topoisomerase associated disease or disorder. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a topoisomerase associated disease or disorder.

The term “infection” includes the pathological state resulting from the invasion of a subject by pathogenic microorganisms, e.g., fungus, bacteria or viruses. Exemplary infections include those caused by the vaccinia family of viruses, malaria, and trypanosomes.

The term “cancer” includes malignancies characterized by deregulated or uncontrolled cell growth, for instance carcinomas, sarcomas, leukemias, and lymphomas. The term “cancer” includes primary malignant tumors, e.g., those whose cells have not migrated to sites in the subject's body other than the site of the original tumor, and secondary malignant tumors, e.g., those arising from metastasis, the migration of tumor cells to secondary sites that are different from the site of the original tumor.

The term “carcinoma” includes malignancies of epithelial or endocrine tissues, including respiratory system carcinomas, gastrointestinal system carcinomas, genitourinary system carcinomas, testicular carcinomas, breast carcinomas, prostate carcinomas, endocrine system carcinomas, melanomas, choriocarcinoma, and carcinomas of the cervix, lung, head and neck, colon, and ovary. The term “carcinoma” also includes carcinosarcomas, which include malignant tumors composed of carcinomatous and sarcomatous tissues. The term “adenocarcinoma” includes carcinomas derived from glandular tissue or a tumor in which the tumor cells form recognizable glandular structures.

The term “sarcoma” includes malignant tumors of mesodermal connective tissue, e.g., tumors of bone, fat, and cartilage.

The terms “leukemia” and “lymphoma” include malignancies of the hematopoietic cells of the bone marrow. Leukemias tend to proliferate as single cells, whereas lymphomas tend to proliferate as solid tumor masses. Examples of leukemias include acute myeloid leukemia (AML), acute promyelocytic leukemia, chronic myelogenous leukemia, mixed-lineage leukemia, acute monoblastic leukemia, acute lymphoblastic leukemia, acute non-lymphoblastic leukemia, blastic mantle cell leukemia, myelodyplastic syndrome, T cell leukemia, B cell leukemia, and chronic lymphocytic leukemia. Examples of lymphomas include Hodgkin's disease, non-Hodgkin's lymphoma, B cell lymphoma, epitheliotropic lymphoma, composite lymphoma, anaplastic large cell lymphoma, gastric and non-gastric mucosa-associated lymphoid tissue lymphoma, lymphoproliferative disease, T cell lymphoma, Burkitt's lymphoma, mantle cell lymphoma, diffuse large cell lymphoma, lymphoplasmacytoid lymphoma, and multiple myeloma.

For example, the therapeutic methods of the present invention can be applied to cancerous cells of mesenchymal origin, such as those producing sarcomas (e.g., fibrosarcoma, myxosarcoma, liosarcoma, chondrosarcoma, osteogenic sarcoma or chordosarcoma, angiosarcoma, endotheliosardcoma, lympangiosarcoma, synoviosarcoma or mesothelisosarcoma); leukemias and lymphomas such as granulocytic leukemia, monocytic leukemia, lymphocytic leukemia, malignant lymphoma, plasmocytoma, reticulum cell sarcoma, or Hodgkin's disease; sarcomas such as leiomysarcoma or rhabdomysarcoma, tumors of epithelial origin such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary-carcinoma, transitional cell carcinoma, chorioaencinoma, semonoma, or embryonal carcinoma; and tumors of the nervous system including gioma, menigoma, medulloblastoma, schwannoma or epidymoma. Additional cell types amenable to treatment according to the methods described herein include those giving rise to mammary carcinomas, gastrointestinal carcinoma, such as colonic carcinomas, bladder carcinoma, prostate carcinoma, and squamous cell carcinoma of the neck and head region. Examples of cancers amenable to treatment according to the methods described herein include vaginal, cervical, and breast cancers in which the tumor cells form recognizable glandular structures.

The language “chemotherapeutic agent” includes chemical reagents that inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and are typically used to treat neoplastic diseases. The chemotherapeutic agents generally employed in chemotherapy treatments are listed below in Table 6. Other similar examples of chemotherapeutic agents include: bleomycin, docetaxel (Taxotere), doxorubicin, edatrexate, etoposide, finasteride (Proscar), flutamide (Eulexin), gemcitabine (Gemzar), goserelin acetate (Zoladex), granisetron (Kytril), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

The language “effective amount” of the compound is that amount necessary or sufficient to treat or prevent a topoisomerase associated disease or disorder. In an example, an effective amount of a compound is the amount sufficient to inhibit undesirable cell growth in a subject, or eliminate a viral or bacterial infection in a subject. The effective amount can vary depending on such factors as the size and weight of the subject, the type of illness, or the particular compound. For example, the choice of the compound can affect what constitutes an “effective amount”. One of ordinary skill in the art would be able to study the factors contained herein and make the determination regarding the effective amount of the compound without undue experimentation.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Materials and Methods

Enzymes and Plasmid DNA. The cloning and purification of wild-type and Y274F vaccinia topoisomerase has been previously described (13). The enzyme concentration was determined by UV absorbance using an extinction coefficient of 28,140 M⁻¹cm⁻¹ in a buffer containing 20 mM sodium phosphate pH 6.0 and 6 M guanidinium hydrochloride (13). Human topoisomerase was obtained from Sigma. The plasmid pUC 19/AID was constructed from pUC19 by inserting the 600 bp gene encoding the enzyme activation induced cytidine deaminase (AID) into the restriction sites NdeI and HindIII.

RNA and DNA Substrates with Fluorescent Tags. The sequence of the 18 mer DNA/RNA substrate containing a single uridine ribonucleotide substitution for the 3′ thymidine residue of the consensus cleavage sequence is shown below, where FAM is 6-carboxyfluorescein,¹ and DAB is the universal fluorescence quencher, dabcyl. For the DNA

18U-FAM/18-DAB
5′ CGTGTCGCCCTUATTCCG-FAM-3′
3′ GGGAAGCGGGAATAAGGC-DAB-5′

cleavage and religation reactions a 18/24 mer duplex was synthesized in which the scissile strand contained two modifications. First, the 5′-end was modified with a FAM label, and second, the

FAM-18AP/24
5′-FAM-CGTGTCGCCCTTPTTCCG-3′
3′-GGGAAGCGGGAATAAGGCTATCAC-5′

nucleotide just 3′ of the ultimate T of the pentameric concensus sequence (underlined) was labeled with the fluorescent adenine analogue, 2-aminopurine (P). These two probes allowed (i) measurement of protein binding by monitoring the increase in fluorescein anisotropy of the DNA, and (ii) DNA cleavage by the increase in 2-aminopurine fluorescence when the 6 mer leaving group rapidly dissociates after strand scission (13). The oligonucleotide strands were synthesized using an ABI 394 synthesizer using nucleoside phosphoramidites obtained from Glen Research. The oligonucleotides were purified using anion exchange HPLC and desalted using disposable gel filtration columns (PD-10, Pharmacia). The purity of oligonucleotides was confirmed using electrophoresis through a 20% polyacrylamide gel containing 7 M urea and MALDI-TOF analysis. The DNA duplexes were prepared in buffer A (20 mM Tris-HCl, 200 mM NaCl, pH 9.0) by mixing the two strands in a molar ratio of 1.1:1 (the dabcyl-labeled strand was in slight molar excess).

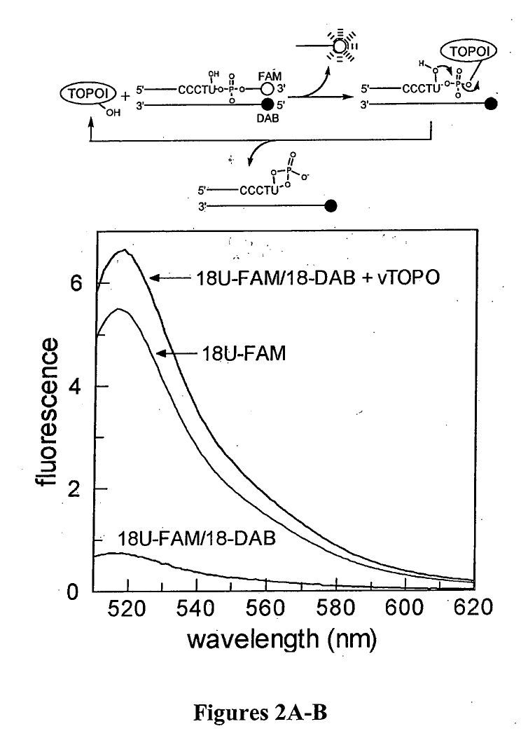

Steady-State Fluorescence Emission Spectroscopy. Fluorescein fluorescence emission spectra of 18U-FAM/18-DAB were collected using a Fluoromax-3 fluorimeter (Instruments S. A. Inc.) at 37° C. The measurements were performed using an excitation wavelength of 492 nm, and the emission was monitored from 510 to 620 nm. The excitation and emission slit widths were set at 0.5 and 5 nm, respectively. Further experimental details are reported in the legend to FIG. 2.

Steady-State Fluorescence Anisotropy Measurements. Samples (150 μL containing 0.1 μM FAM-18AP/24) were excited with vertically polarized light at 492 nm (1-nm bandpass), and both vertical and horizontal emission was monitored at 517 nm (3-nm bandpass). Three replicate measurements were made for each addition of topoisomerase in the range 0 to 2.5 μM, and the values were then averaged. The G factor was calculated and its value was used to calculate the anisotropy. The equilibrium dissociation constant was calculated by nonlinear-least squares fitting of the data to eq 1, where A is the fluorescence anisotropy at a given concentration of vTopo, Aₒ and A_{f} are the anisotropies of the free DNA and the vTopo-DNA complex, and [E]ₜₒₜ

$$A = {A}_{0} - ( ( {A}_{0} - {A}_{f} ) / {2 [ DNA ]}_{tot} \{ b - \sqrt{{b}^{2} - {{4 [ E ]}_{tot} [ DNA ]}_{tot}} \} ) \qquad ( 1 )$$

and [DNA]ₜₒₜ are the total concentrations of DNA and vTopo. To determine whether 112983 displaced the bound DNA from Y274F, a complex was formed using 0.1 μM FAM-18AP/24 and 2 μM Y274F, and the anisotropy was measured as increasing concentrations of 112983 were added to the solution (0 to 28 μM).

Presteady-State Kinetic Measurements of vTopo RNase Activity. Reactions (150 μL) were performed in buffer A at 37° C. using 10 nM vTopo and substrate concentrations of 0.05, 0.1, 0.2, 0.4 and 0.8 μM. Reactions were initiated by the addition of 2 μL of a concentrated vTopo solution to the 18U-FAM/18-DAB substrate and buffer. After mixing, the increase in fluorescence intensity at 517 nm with excitation at 492 nm was followed for 3600 s. The excitation and emission slit widths were set at 0.5 and 5 nm, respectively. Time points were collected at 1 s intervals with an integration time of 1 s. Raw fluorescence readings were converted to product concentration using eq 2, and the data were fitted by computer simulation to the mechanism shown in Scheme 1 (see Results and Discussion) using the program Dynafit (14). In this equation Fₒ is the initial fluorescence, Fₜ is the measured fluorescence at time t, and Fₘₐₓ is the fluorescence after complete conversion of substrate to product.
[P]ₜ=[S]ₜₒₜ×(Fₜ−Fₒ)/(Fₘₐₓ−F) (2)

High-Throughput Inhibition Studies. For screening of the NCI Diversity Library, a Fluoromax-3 fluorimeter with a Micro-Max 96-well fluorescence plate reader attachment was utilized. In our initial studies, five plates from the library were screened, comprising 480 unique compounds. Each reaction well in the screen contained enough test compound to give a 100 μM solution after addition of 200 μL of reaction solution containing enzyme (10 nM) and buffer A. Reactions were then initiated by the addition of 18U-FAM/18-DAB (0.5 μM final concentration in the well). For validation, positive and negative control wells were also included that consisted of enzyme and substrate without inhibitor, and substrate without enzyme. The rates of fluorescence change at 517 nm with excitation at 492 nm were followed by taking three fluorescence readings in each well over a 30 minute time period.

Inhibition of the vTopo RNase Activity by 112983. To determine the inhibition mechanism of the best inhibitor identified, 112983, the concentration dependence of RNase inhibition was studied at two concentrations of 18U-FAM/18-DAB equal to about ⅙ and 10 times the Kₘ. These two substrate concentrations allow determination of whether the inhibition is competitive, or alternatively, involves a ternary complex between the enzyme, substrate and inhibitor. The [112983] was varied in the range 0 to 32 μM, and the fluorescence changes as a function of time were monitored using the MicroMax plate reader as described above. Global kinetic fitting of the product formation curves was used to determine the Kᵢ for inhibition by 112983 using the program Dynafit (14).

Inhibition of Steady-State Plasmid Supercoil Relaxation. Supercoil relaxation reactions were performed with 100 nM supercoiled pUC19/AID, 3 nM vTOPO and various concentration of inhibitor. The reaction mixtures were incubated for 9 minutes at room temperature and were then quenched with one volume of loading buffer containing 0.4% SDS, 5% glycerol and 2×TBE. The relaxed DNA and supercoil DNA were resolved on 1% agarose gel at 75 V for 1.5 hours in a running buffer consisting of 50 mM Tris, 160 mM glycine, pH 7.5. After ethidium bromide staining and fluorescence imaging using a BioRad GelDoc instrument, the bands corresponding to supercoiled and relaxed DNA in each lane were quantified using the software supplied with the instrument. Data were plotted as fraction relaxed DNA [counts relaxed DNA/(counts relaxed DNA+counts supercoiled DNA)] against [112983].

Single-Turnover Cleavage Reactions. Inhibition of single-turnover cleavage was investigated using the 2-aminopurine labeled DNA duplex (FAM-18AP/24) (15). The reactions were performed using an Applied Photophysics stopped-flow fluorescence instrument in the two-syringe mode. Equal volumes of 1 μM vTopo and 50 nM DNA in the presence or absence of 8 μM 112983 were rapidly mixed, and the increase in 2-aminopurine fluorescence was followed using excitation at 315 nm with monitoring at emission wavelengths greater than 360 nm using a cut off filter (13).

Single-Turnover Religation Reactions. Experiments were performed by rapidly mixing a preformed covalent complex with an excess of a 12-mer ligation strand (5′ ATTCCGATAGTG 3′). The covalent complex (E-FAM-12/24-mer) was formed by incubating topoisomerase (236 nM) with FAM-18AP/24 (200 nM) for 15 min. The religation reactions (20 μL total volume) were initiated by rapid mixing of equal volumes of the covalent complex with the complementary 12-mer strand, and the reactions were quenched after 10 s by the addition of loading buffer containing 4% SDS. The final concentrations of the covalent complex and 12-mer were 200 nM and 2.7 μM, respectively. Reactions were performed both in the presence and absence of 32 μM 112983. The E-FAM-12/24 mer covalent complex and the FAM-24/24-mer religation product were separated by electrophoresis using a 15% (w/v) polyacrylamide gel containing SDS. The fractional extent of covalent complex remaining at a given time (frac complex=cpm complex/total cpm) was quantified using fluorescence imaging with the Image Quant software package provided with the instrument (Typhoon 9410, Amersham Biosciences).

Equilibrium Cleavage Measurements. The equilibrium cleavage reactions were performed in the presence and absence of compound 112983 (100 μM) essentially as described previously (13). Wild-type topoisomerase (360 nM) and 5′-³²P-labeled 40/40-mer DNA (300 nM) with the scissile strand sequence of 5′ AACATATCCGTGTCGCCCTTATTCCGATAGTGACTACAGC 3′, were incubated for five minutes at room temperature and the covalent complex present at equilibrium was trapped by the rapid addition of 1 volume of 5% SDS (20 μl). The reactions in the presence of 112983 were performed by preincubating the compound with the enzyme or DNA for five minutes before initiating the reaction. The free and covalently bound DNA were separated by electrophoresis using an 15% polyacrylamide gel containing 0.2% SDS. The fraction covalent complex was then determined [Frac complex=counts covalent complex/(counts covalent complex+counts free DNA)] by phosphorimaging.

### Results and Discussion

A Continuous Multiple-Turnover Kinetic Assay for Topo I. A molecular beacon substrate was constructed that contained a 6-carboxyfluorescein group (FAM) on the 3′ end of the scissile strand and a dabcyl (DAB) quench attached to the 5′ end of the complementary strand (FIG. 2). Cleavage of the scissile strand by topo I was expected to rapidly release the FAM labeled 6 mer strand to solution, thereby relieving the strong fluorescence quench provided by the DAB group. For this kinetic assay to be mechanistically useful, the release of the 6 mer FAM strand needs to be fast compared to strand cleavage and 2′,3′ cyclic phosphodiester formation. It has been previously established that dissociation of an identical 6 mer DNA strand is much faster than the cleavage rate for a DNA substrate containing a CCCTT sequence (k_{cl}=0.7 s⁻¹) (13). As will be shown below, cleavage of a ribonucleotide phosphodiester is 70 times slower than for the preferred DNA substrate, indicating that 6 mer strand dissociation is also not rate-limiting for the RNase reaction.

We investigated the magnitude of the fluorescence increase that occurs upon topo I cleavage of the molecular beacon substrate. Prolonged incubation of one micromolar substrate with 0.2 μM topo I resulted in a 9-fold increase in fluorescence at 520 nm (FIG. 2). The fold increase in fluorescence at the completion of the reaction was found to be similar to the FAM-labeled single stranded DNA (18U-FAM, FIG. 2), independent of topo I concentration in the range 20 to 1000 nM, and independent of DNA concentration in the range 0.1 to 3 μM (not shown). These results indicate that there is a linear relationship between the change in fluorescence and the amount of product formed over a reasonable range of reaction conditions, and that the fluorescence measurements can be used quantitatively in mechanistic studies.

Presteady-State Kinetic Analysis of Topo I Ribonuclease Activity. The ribonuclease reaction of topo I occurs by a minimum of three steps involving substrate binding and dissociation to form an ES complex with the rate constants kₒₙ and k_{off}, attack of the tyrosine nucleophile to generate a phosphotyrosine intermediate E-I with a rate constant k_{cl}, and release of the enzyme by attack of the 2′ hydroxyl of the uridine ribonucleotide (kₘ) to produce the 2′,3′ cyclic phosphodiester product as shown in Scheme 1.

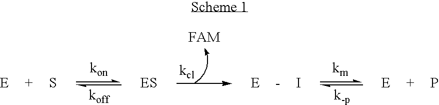

Although attack of the 5′ OH leaving group could kinetically compete with kₘ, dissociation of the small 6 mer strand is rapid compared to the previously measured religation rate constant (kᵣ=1 s⁻¹) (13). Thus, rapid strand dissociation makes the cleavage step essentially irreversible under these conditions. The final rate constant in Scheme 1 (k₋ₚ) takes into account that product (P) can accumulate during the reaction assay and may act as a competitive inhibitor with respect to substrate binding to the enzyme. In a presteady-state kinetic study, a burst of product formation will be observed when a stable intermediate is formed prior to a slower step on the reaction pathway (16). Under such conditions, the substrate concentration dependence and amplitude of the exponential burst phase provide information on the binding and dissociation rate constants to form ES, as well as the rate constant for formation of the covalent intermediate, E-I. The subsequent linear rate provides information on the rate-limiting turnover of E-I to form free enzyme and product. In contrast, if the rate of intermediate formation is comparable to the rate of the subsequent steps the burst will be attenuated, and if product inhibition is pronounced, a linear steady state region will be difficult to measure.

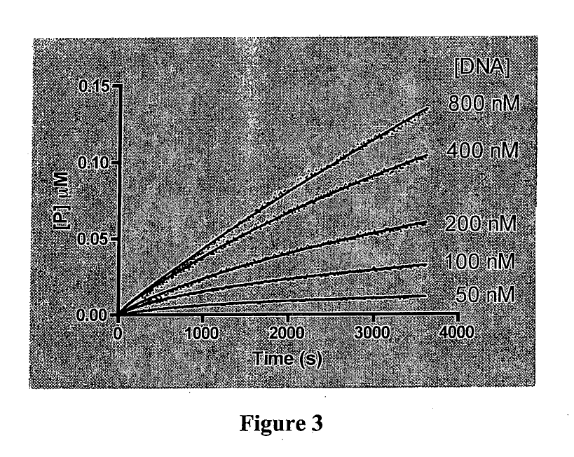

The topo I ribonuclease reaction lacks a clear presteady-state burst and linear steady-state region, indicating that the rate of formation of the phosphotyrosine intermediate is comparable to the rate of attack of the 2′ hydroxyl group, and that product inhibition is significant (FIG. 3). Given the complexity of the kinetic time courses, the data in FIG. 3 were fitted by computer simulation to the mechanism shown in Scheme 1 to provide each of the four rate constants as reported in Table 1.

TABLE 1
Kinetic parameters for the topo I RNase activity^{a}

| K_{D} | kₒₙ | k_{off} | k_{cl} | kₘ | k₋ₚ |
|---|---|---|---|---|---|
| (μM) | μM⁻¹s⁻¹) | (s⁻¹) | (s⁻¹) | (s⁻¹) | (μM⁻¹s⁻¹) |
| 0.44 | 16 | 7 | 0.008 | 0.008 < k₋ₚ < 0.017 | 0.5 < k₋ₚ < 4 |

^{a}Kinetic parameters were determined at 25° C. in buffer A. Errors for K_{D}, kₒₙ, k_{off}, and k_{cl} were less than ±15%. Only a range of values for kₘ and k₋ₚ were defined by the data, because kₘ is comparable to or greater than k_{cl}, and product inhibition was modest.

Scheme 1 was found to be the simplest model that fit the entire data. The association and dissociation rate constants are similar to the previously reported values for the all DNA cleavage reaction (kₒₙ =16 μM⁻¹ s⁻¹ and k_{off}=7 s⁻¹) (13). However, the rate constant for attack of the tyrosine nucleophile is 70-fold slower than for an all DNA substrate (k_{cl}=0.008 s⁻¹ for RNA versus k_{cl}=0.7 s⁻¹ for DNA) (13). Because the assay detects the cleavage and fast release of the FAM-6 mer, the apparent rate constants for the subsequent ribonuclease and product binding reactions were poorly determined by the data. Rate constants for these reactions fell in the range kₘ=0.008 to 0.017 s⁻¹ and k₋ₚ=0.5 to 4 μM⁻¹ s⁻¹ in the individual simulations of the data shown in FIG. 3.

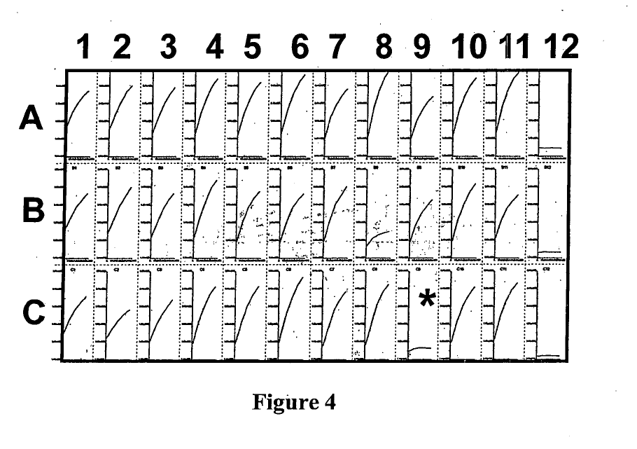

Screening the NCI Diversity Library for Topo I Inhibitors. As a test of the molecular beacon assay in a high-throughput application we screened about one-quarter of the 2000 member Diversity Library available from the NCI. This library consists of small molecules that are selected based upon a defined set of diversity criteria, and are expected to probe chemical space in an efficient manner. A 96-well format was used in which each well contained 10 nM vTopo, 0.5 μM substrate, and 100 μM library compound (see Material and Methods), and representative data acquired from three rows of plate 3846 is shown in FIG. 4. Using these conditions, a total thirteen compounds were identified that inhibited vTopo activity by greater than 50% (Table 2).

TABLE 2
Compounds identified from the NCI
Diversity Library as vTopo inhibitors.

| | | % vTopo |
|---|---|---|
| NSC Number | Structure | activity^{a} |
| 112983 | 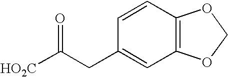 | 9 |
| 125214 | 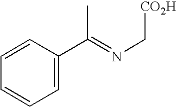 | 53 |
| 2805 | 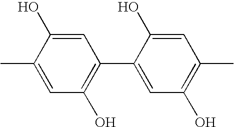 | 30 |
| 150982 | 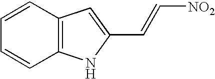 | 54 |
| 75600 | 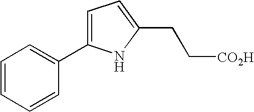 | 35 |
| 39215 | 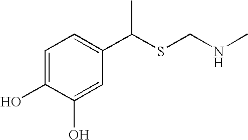 | 27 |
| 39225 | 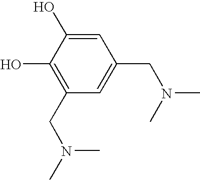 | 52 |
| 163339 | 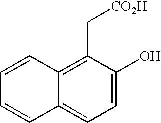 | 48 |
| 120917 | 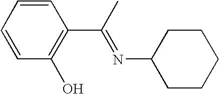 | 28 |
| 379552 | 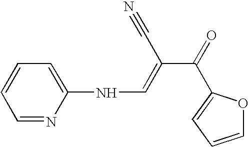 | 7 |
| 14555 | 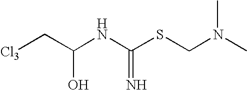 | 28 |
| 403376 | 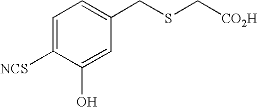 | 45 |
| 7962 | 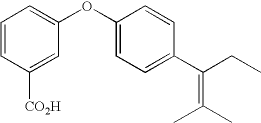 | 33 |

^{a}Approximately 500 compounds from the library were screened. The screen was performed at [S]˜Kₘ and [I] = 100 μM. The % activity is compared to that of the enzyme and substrate in the absence of inhibitor. Only compound 112983 was validated in secondary screens and by obtaining an authentic sample from the NCI repository. The other compounds were not subjected to further validation.

In addition to those compounds identified in Table 2, the inhibitory compounds listed in Table 3 have been discovered in further screening of the NCI Diversity Library against the vaccinia virus topoisomerase I target. Thirteen of the most potent compounds were also tested using a standard plasmid supercoil release assay to ascertain that the inhibition also extended to large DNA substrates (31).

TABLE 3
Newly discovered inhibitors of vaccinia virus type I topoisomerase

| | | | | % |
|---|---|---|---|---|
| | | | | Inhibition |
| | | NCI | % | (plasmid |
| | | Plate | Inhibition | SC |
| | NSC | ID-well | (RNAse | release |
| Chemical Structure | Number | position | HTS)* | assay)** |
| 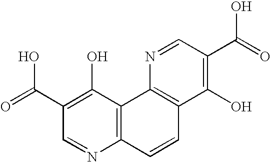 | 1288884 | 3851-G3 | Partial @100 μM | NT*** |
| 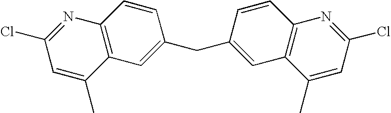 | 109451 | 3857-D4 | Partial @100 μM | NT |
| 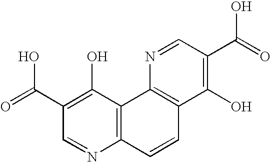 | 77552 | 3857-E7 | Partial @100 μM | NT |
| 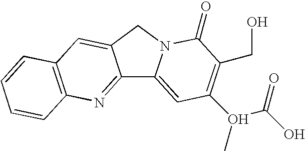 | 100880 | 3857-F4 | Partial @100 μM | NT |
| 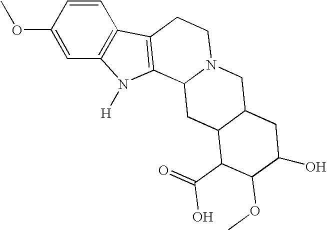 | 81463 | 3857-F5 | Partial @100 μM | NT |
| 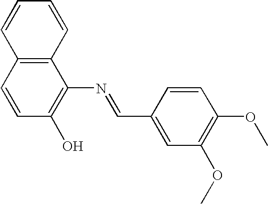 | 17061 | 3852-F5 | Partial @100 μM | NT |
| 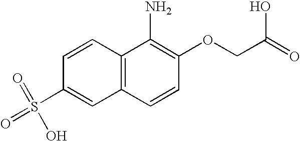 | 16163 | 3848-A2 | 100% @100 μM | NT |
| 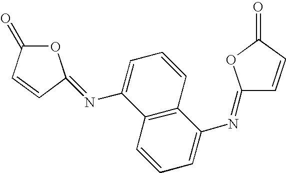 | 69575 | 3852-C7 | 100 % @100 μM | NT |
| 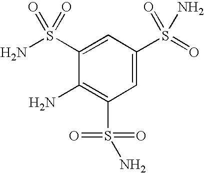 | 25857 | 3853-G4 | 100% @100 μM | NT |
| 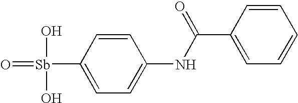 | 15596 | 3857-C11 | 100% @100 μM | NT |
| 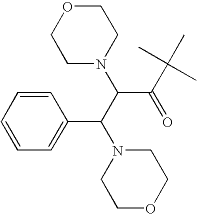 | 33571 | 3858-A11 | 100% @100 μM | NT |
| 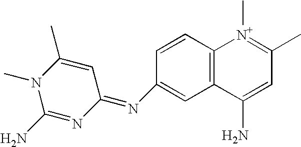 | 48223 | 3858-H2 | 100% @100 μM | NT |
| 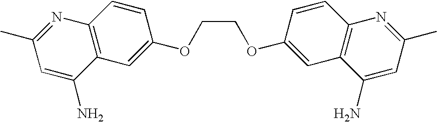 | 22907 | 3860-B9 | 100% @100 μM | NT |
| 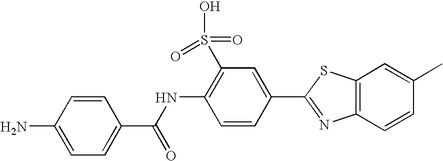 | 26699 | 3860-D10 | 100% @100 μM | NT |
| 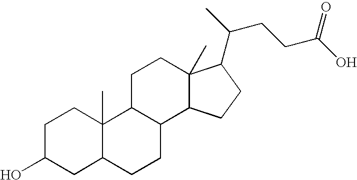 | 683770 | 3860-E6 | 100% @100 μM | NT |
| 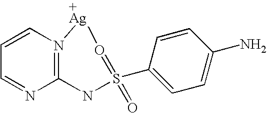 | 625324 | 3860-E2 | 100% @100 μM | NT |
| 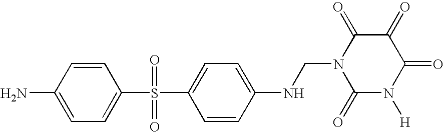 | 34238 | 3860-E3 | 100% @100 μM | NT |
| 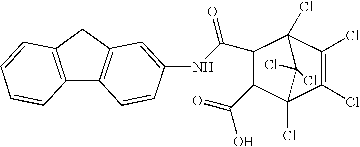 | 73101 | 3864-A5 | 100% @100 μM | NT |
| 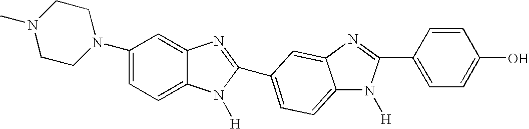 | 322921 | 3864-B8 | 100% @100 μM | NT |
| C₁₀H₁₀Cl₄N₁₀Pt | 177395 | 3864-C4 | 100% @ | NT |
| | | | 100 μM | |
| 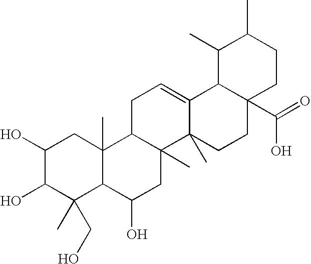 | 88135 | 3864-D6 | 100% @100 μM | NT |
| 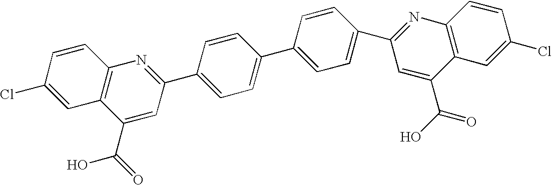 | 210627 | 3864-E4 | 100% @100 μM | NT |
| 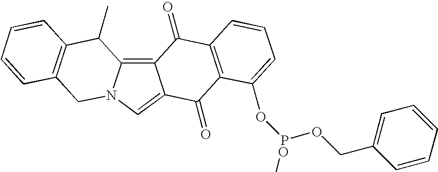 | 611615 | 3864-B9 | 100% @100 μM | NT |
| C₁₀H₂₂N₂O₂Cl₆Pt | 295558 | 3864-B11 | 100% @ | NT |
| | | | 100 μM | |
| 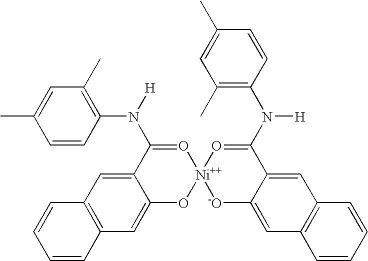 | 306698 | 3864-D3 | 100% @100 μM | NT |
| 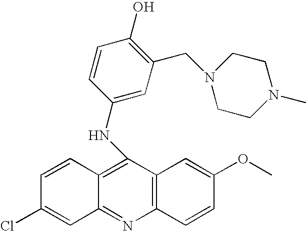 | 130813 | 3864-D7 | 100% @100 μM | NT |
| 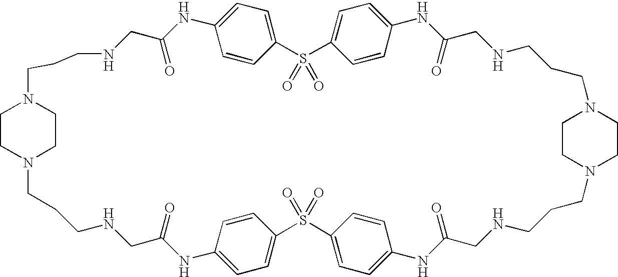 | 95609 | 3865-C9 | 100% @100 μM | NT |
| 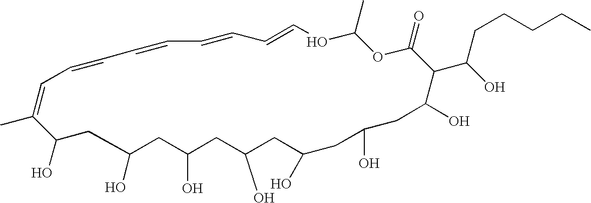 | 3364 | 3865-D9 | 100% @100 μM | NT |
| 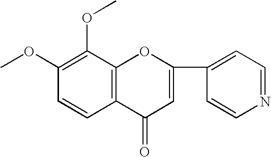 | 54646 | 3849-F6 | 100% @10 μM | NT |
| 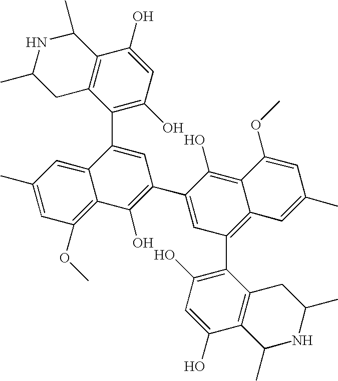 | 661755 | 3865-A2 | 100% @10 μM | NT |
| C₂₁H₈Cl₁₂O₅S | 270718 | 3865-A4 | 100% @ | NT |
| | | | 10 μM | |
| 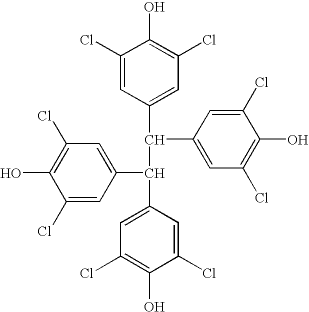 | 172033 | 3865-B5 | 100% @10 μM | NT |
| 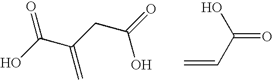 | 146443 | 3865-F5 | 100% @10 μM | NT |
| 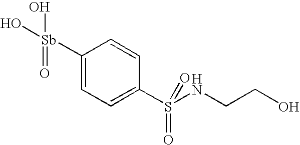 | 13776 | 3857-D5 | 100% @5 μM | 100% @10 μM |
| 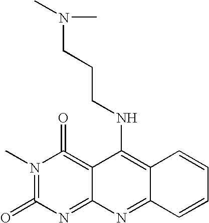 | 373989 | 3860-G4 | 100% @5 μM | 70% @0.5 μM |
| 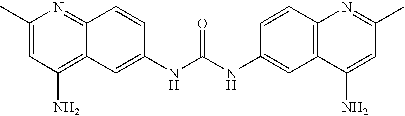 | 12155 | 3860-H7 | 100% @2.5 μM | 40% @0.5 μM |
| 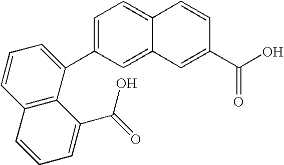 | 7810 | 3852-H11 | 100% @2.5 μM | 100% @10 μM |
| 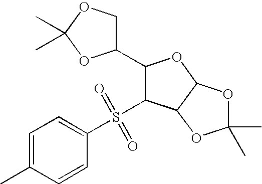 | 14163 | 3860-E8 | 100% @2.5 μM | 50% @5 μM |
| 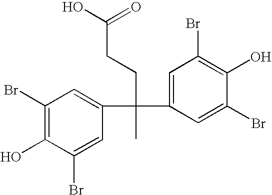 | 21588 | 3864-E9 | 100% @1.2 μM | 100% @5 μM |
| 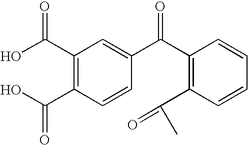 | 28620 | 3853-H5 | 100% @1.0 μM | 100% @5 μM |
| 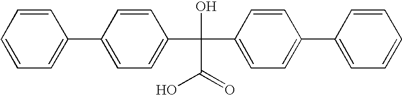 | 28086 | 3857-B2 | 100% @1.0 μM | 100% @5 μM |
| 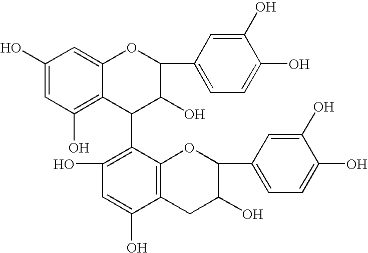 | 143099 | 3865-F4 | 100% @1.0 μM | 100% @5 μM |
| 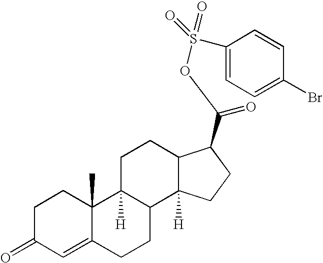 | 88915 | 3864-E6 | 100% @0.5 μM | 50% @0.006 μM |
| 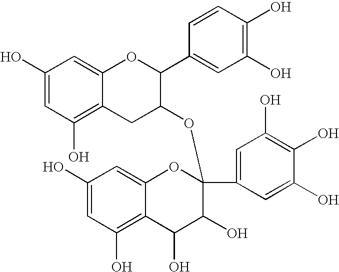 | 143101 | 3864-E10 | 100% @0.5 μM | 70% @0.5 μM |
| 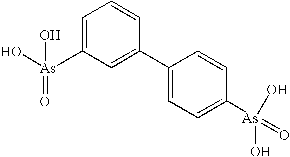 | 48300 | 3860-A2 | 100% @0.1 μM | 50% @0.006 μM |
| 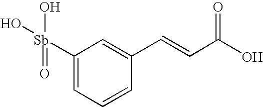 | 13778 | 3852-D5 | 100% @0.1 μM | 70% @0.006 μM |

*The percent inhibition observed by the given compound as determined using the high-throughput screening invention previously described (31).
**Inhibition observed in standard plasmid supercoil (SC) release assay (31).
***NT, not tested

Many of the hits showed similar structural features consisting of a substituted phenyl ring that often contained one or more hydroxyl groups. An additional motif that appeared was two fused ring structures, or two rings connected by a single or multiple bridging atoms. Two of the compounds (112983 and 379552, see Table 2) showed greater than 90% inhibition under these reaction conditions, and compound 112983 was subjected to further mechanistic analysis.

Selectivity Studies

To ascertain the selectivity of the four most potent hits for the vaccinia target, these were also tested for inhibition of the human type IB topoisoinerase using a standard supercoil release assay (Table 4) (31). All of these compounds showed 800 to >3,000-fold stronger inhibition of the vaccinia enzyme than for the human type I topoisomerase, indicating that these are vaccinia virus-specific topoisomerase poisons that may be useful leads in developing anti-vaccinia virus drugs.

TABLE 4
Potency of Selected Compounds Against
the Human Type IB Topoisomerase

| NSC | NCI Plate ID-well | % Inhibition (plasmid SC | |
|---|---|---|---|
| Number | position | release assay)** | Selectivity* |
| 13778 | 3852-D5 | 100% @ 5 μM, | ˜800 |
| | | 0% @ 2 μM | |
| 48300 | 3860-A2 | 50% @ 20 μM | 3333 |
| 14163 | 3860-E8 | 0% @ 20 μM | >3333 |
| 88915 | 3864-E6 | 50% @ 5 μM | 833 |

*Estimated as the ratio of the concentrations of compound that gave similar levels of inhibition for the vaccinia and human enzymes in the supercoil release assay
(i.e. [I]^{human}/[I]^{vaccinia}).

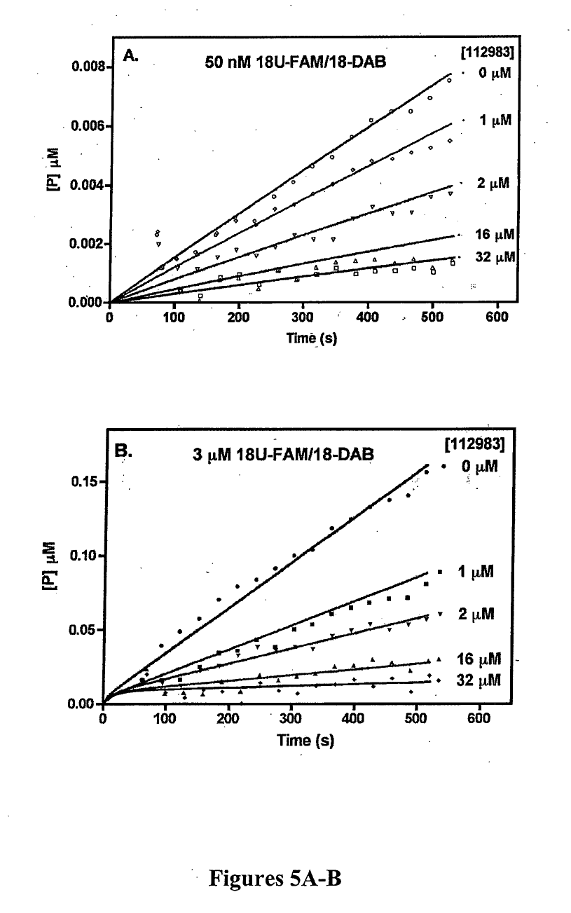

Mechanism of RNase Inhibition by 112983. To further investigate the mechanism of inhibition, we performed inhibition studies using conditions where the 18U-FAM/18-DAB substrate concentration was much less than and greater than its Kₘ. The rationale for these conditions is that a competitive inhibitor will show a large increase in its apparent Kᵢ when the assay is performed at high substrate concentrations due to competitive binding effects, whereas if inhibition involves the formation of a ternary complex between E, S and inhibitor (i.e. noncompetitive or uncompetitive inhibition), the fractional inhibition will be insensitive to substrate concentration. The initial rates of product formation starting from 50 nM and 3 μM 18U-FAM/18-DAB substrate in the presence of increasing concentrations of 112983 are shown in FIG. 5A and 5B, respectively. For both substrate concentrations, 50% inhibition was observed at about 2 μM 112983, even though the substrate concentrations differed by 60-fold in these two reactions. These results are consistent with an uncompetitive mode of inhibition, requiring that 112983 binds to the covalent complex (E-I, Scheme 2). An alternative noncompetitive 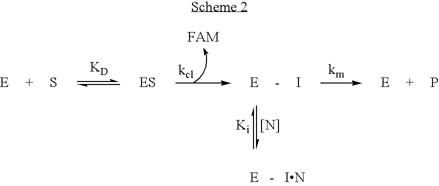
mechanism in which N binds to the ES complex would be expected to increase the affinity of S for the enzyme by a mass action effect, which was not observed in trial simulations of the progress curves (not shown)(17). Thus we simulated the progress curves shown in FIG. 5 using the simplest mechanism in which 112983 (N, Scheme 2) binds to the E-I covalent complex. This mechanism is most consistent with the data because binding of N occurs after irreversible formation of the E-I complex, and therefore, does not affect binding of S. From these simulations (solid lines, FIG. 5), we determined a Kᵢ=1.6±0.6 μM for 112983 using the predetermined kinetic constants for the substrate reported in Table 1.

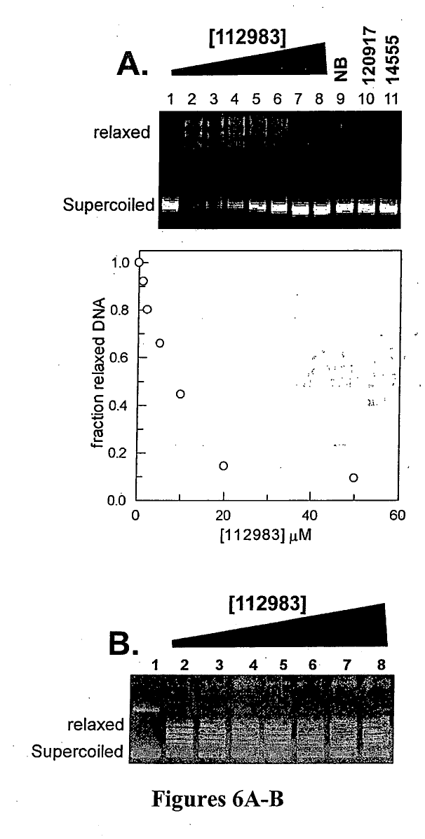

Inhibition DNA Supercoil Relaxation by vTopo and hTopo. An important question with using the ribonuclease activity to uncover inhibitors is whether the inhibition also extends to the biologically relevant DNA reactions of the enzyme. To address this question we tested whether 112983 inhibited plasmid supercoil unwinding by vTopo as well as the closely related human topoisomerase IB (hTopo). As shown in FIG. 6A, increasing concentrations of 112983 brought about complete inhibition of the supercoil relaxation activity of vTopo. Although these reactions are not carried out under initial rate conditions, 50% inhibition was observed at a concentration of 112983 of about 7 μM. This concentration is similar to the value required to inhibit the ribonuclease reaction by 50%. We also investigated the inhibition of supercoil relaxation by two other compounds in the NCI library (120917 and 14555, see Table 2), as well as the weak natural product inhibitor of vTopo, novobiocin (FIG. 6A, lanes 9-11)(18). Each of these compounds brought about complete inhibition of the relaxation reaction when present at concentrations 500 to 1000 μM (FIG. 6A). We conclude that the ribonuclease assay reliably detects compounds that are inhibitory to the biologically relevant DNA relaxation reaction.

To investigate the specificity of the observed inhibition, we tested whether the inhibition also extended to supercoil relaxation catalyzed by hTopo (FIG. 6B). In contrast to the results with vTopo, increasing the concentration of compound 112983 from 0 to 32 μM resulted in no detectable inhibition of the hTopo reaction. This result excludes the possibility that 112983 is an indiscriminant protein poison, and instead, indicates that the inhibition by 112983 takes advantage of structural or mechanistic differences between vTopo and hTopo.

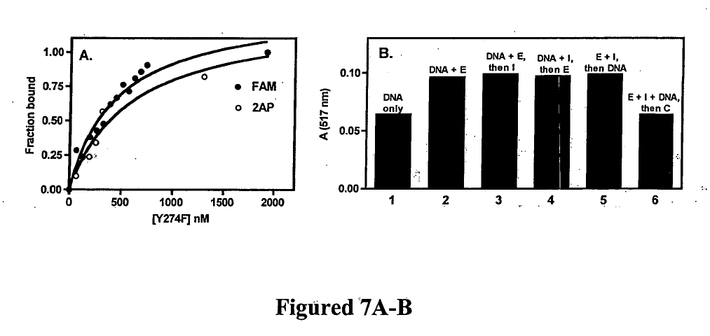

Does 112983 Inhibit DNA Binding? The observed inhibition of the RNase and DNA supercoil relaxation activities suggests that binding of compound 112983 is not competitive with the DNA substrate (see above). To confirm these results using another approach, we investigated whether 112983 inhibited noncovalent binding by Y274F vTopo, which lacks the active site tyrosine nucleophile. In this analysis, we followed the change in fluorescence anisotropy upon Y274F binding to the 5′-FAM 18AP/24 duplex (FIG. 7A). Addition of increasing concentrations of Y274F to a solution of 5′-FAM 18AP/24 results in a hyperbolic increase in the steady-state fluorescence anisotropy of the DNA (FIG. 7A, closed circles), as would be expected upon the formation of a high molecular weight enzyme-DNA complex. The data were fitted to a simple one-site binding equation (eq 1) from which a K_{D} value of 500±60 nM was calculated. A similar binding constant was determined by following the increase in fluorescence of the 2-aminopurine base (open circles, FIG. 7A) (K_{D}=700±200). These binding constants are similar to the previously measured K_{D} for an identical duplex that lacked the 5′-FAM group, establishing that this label does not interfere with the noncovalent interaction of Y274F with the DNA.

To determine if binding of 112983 was competitive with the DNA, we formed a complex between Y274F and 5′-FAM 18AP/24, and monitored the anisotropy as in the presence of 112983 (FIG. 7B). At a concentration of compound as high as 32 μM, the anisotropy was unchanged from that of the enzyme-DNA complex alone (FIG. 7B, compare columns 2 and 3). If binding of the compound had displaced the bound DNA, the anisotropy should have returned to the level of the free DNA (FIG. 7B, column 1). Changing the order of addition of enzyme, compound and DNA did not result in a decrease in anisotropy, excluding the possibility that slow onset binding by the inhibitor was influencing the results (FIG. 7B, columns 4 and 5). As a positive control, we also added 3 μM of nonfluorescent competitor DNA to the sample containing the complex and compound, and observed that the anisotropy returned to the level of the free DNA (FIG. 7B, column 6). These data clearly establish that 112983 does not inhibit substrate binding by vTopo.

Does 112983 Inhibit Single-Turnover DNA Strand Cleavage and Religation? The two half-reactions of vTopo (DNA cleavage and religation) can be studied separately using single-turnover conditions. In the cleavage experiment, a “suicide” DNA substrate is employed in which the strand containing the 5′ OH leaving group is sufficiently small that it rapidly dissociates upon cleavage of the phosphodiester linkage (15), resulting in irreversible formation of the covalent complex. If the leaving strand contains a 2-aminopurine fluorescent label on the 5′ end, rapid strand dissociation leads to an increase in the 2-aminopurine fluorescence that is a measure of the preceding rate-limiting cleavage reaction (13). In the religation experiment, the covalent complex is first formed using a suicide substrate and then the ligation reaction is initiated by the addition of a large excess of a complementary DNA strand containing a 5′ OH nucleophile. The extent of ligation may be assessed by electrophoretically separating the covalent complex and ligated DNA using SDS-PAGE (15).

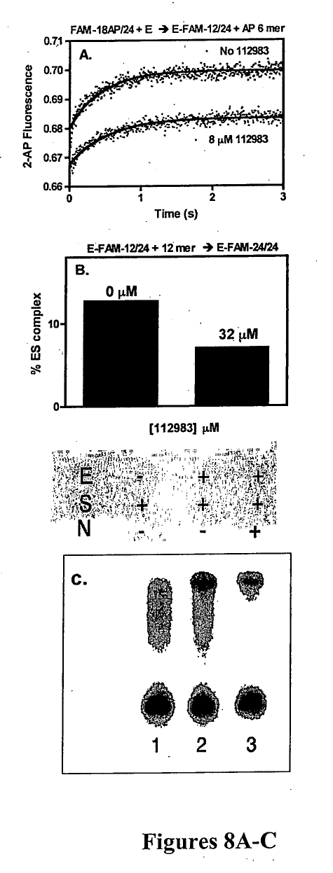

We performed stopped-flow fluorescence analysis of the single-turnover cleavage reaction of the FAM-18AP/24 suicide substrate in the absence and presence of 8 μM 112983, a concentration that would result in about 75% inhibition of the ribonuclease reaction rate (FIG. 8A). The cleavage rate in the absence of compound was 2.3 s⁻¹, which is identical to that previously observed for the 18AP/24 mer that lacked the FAM group (13). The presence of 112983 had no effect on the single-turnover cleavage rate (FIG. 8A, lower curve), indicating that inhibition of the steady-state reactions involves a step after formation of the covalent complex. As observed for DNA binding, the results were the same regardless of whether the compound was preincubated with the enzyme or DNA (not shown).

For the single-turnover religation reaction, the covalent complex between vTopo and the FAM-18AP/24 substrate was rapidly mixed with a 12 mer strand in the absence and presence of 112983 (FIG. 8B). Ten seconds after initiation the reactions were quenched and the fraction covalent complex remaining was determined by separating the complex and DNA ligation product by gel electrophoresis followed by fluorescence imaging. The fraction covalent complex remaining at the ten second quench time was decreased by 50% in the presence of 16 μM 112983, suggesting that this compound alters the cleavage equilibrium (K_{cl}) by increasing kᵣ (K_{cl}=[covalent complex]/[noncovalent complex]=k_{cl}/kᵣ) (FIG. 8B). In this analysis, we could have detected a rate decrease of 6-fold given the known half-life of the religation reaction (˜0.7 s⁻¹)(13). As noted below, an increase in the rate of religation caused by 112983 would not be detected in this assay, but would appear as a decrease in the equilibrium endpoint because K_{cl}=[covalent complex]/[noncovalent complex]=k_{cl}/kᵣ. Thus, the results are consistent with a modest increase in the religation rate induced by 112983.

We also further explored whether 112983 affected the overall cleavage equilibrium by performing equilibrium cleavage measurements. In these reactions, vTopo reacts reversibly with a 40/40 mer substrate that has a 5′-³²P label on the scissile strand (15). The equilibrium reaction is rapidly quenched by the addition of SDS which traps the covalently bound enzyme. FIG. 8C shows the extent of equilibrium trapping of covalently bound vTopo in the presence and absence of 100 μM 112983. In the presence of compound we noted a 2-fold decrease in the amount of covalent complex as compared to a control reaction in the absence of compound. Since 112983 does not inhibit single-turnover cleavage (see above), the observed decrease in the amount of covalent complex formed at equilibrium is consistent with an increase in the rate of religation. An increase in the DNA strand ligation rate would act to slow the steady-state RNase and plasmid supercoil unwinding reactions by decreasing the concentration of the reactive intermediate. However, this effect cannot explain the entire inhibitory behavior of this compound because a finite rate would be expected at saturating concentrations of compound, yet the steady-state reactions are completely inhibited (see below).

Mechanism of Inhibition. The above findings indicate that 112983 follows a novel inhibitory mechanism for type IB topoisomerases. This small molecule selectively inhibits the steady-state RNase and supercoil unwinding reactions of vTopo but not hTopo, and yet remarkably, exhibits no detectable inhibition of DNA binding, or the single-turnover half reactions of cleavage and religation (FIGS. 7 and 8). These observations eliminate two simple mechanisms of inhibition such as competition with substrate binding, or targeting of the cleavage chemistry via the formation of an inhibitory ESN complex (where N is 112983). A viable mechanism that is consistent with all of the data is one in which 112983 targets the E-I covalent complex and inhibits attack of the 2′ OH in the ribonuclease reaction, and supercoil unwinding with the plasmid substrate (Scheme 3). According to this mechanism, at infinite concentration 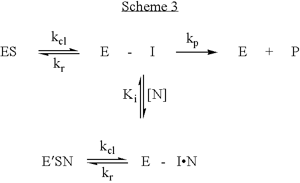
of 112983, the equilibrium is shifted completely to the inhibitory EIN complex, which is in equilibrium with the E′SN complex. Thus, cleavage and religation are unaffected but 2′ OH attack and supercoil unwinding are thwarted. The inhibitory mechanism likely involves modest stimulation of the religation rate (FIG. 8B, 8C), which would serve to decrease the lifetime of the covalent complex. Shortening the lifetime of the covalent E-I complex inhibits the steady-state RNase and supercoil release reactions because attack of the 2′ OH and strand rotation are competitive with 5′ OH attack (7). A more complicated mechanism that involves binding of 112983 to the E or ES species is not consistent with the data because such binding would lead to an increase in the apparent affinity of S for the enzyme through mass action effects, which is not observed (17). Thus, formation of the noncovalent ES complex follows an ordered mechanism in which 112983 first binds to E-I to form E-IN, and then E-N is converted to E′SI (Scheme 3). The data do not distinguish whether 112983 exerts its effect by direct interaction at the cleavage site or through binding to an allosteric site. An allosteric mechanism is not unprecedented with vTopo, as nucleotide triphosphates and inorganic pyrophosphate have been found to dramatically stimulate the DNA supercoil relaxation activity of vTopo (19). It is quite possible that the stimulation of DNA ligation by 112983 also arises from an allosteric mechanism.

Comparison with Other Topo I Inhibitors. Type IB topoisomerases can be targeted at multiple points along their reaction pathways ranging from the noncovalent DNA binding step (18, 20), the attack of the tyrosine nucleophile (9, 21, 22), the reverse attack of the 5′-OH (23), and the supercoiled unwinding step (9). Previous studies have shown that DNA binding by vTopo is competitively inhibited by two structurally related antibiotic molecules, novobiocin and coumermycin (18). The inhibition by these molecules is surprising given that they are potent inhibitors of bacterial DNA gyrase enzymes, where they act by binding to the ATP binding sites of these enzymes (24, 25). Like 112983, novobiocin shows inhibition of vTopo (Kᵢ=325 μM), but no inhibition of the related human topo IB enzyme (7). Other natural product inhibitors of vaccinia virus type IB topoisomerases have been identified (20, 23, 26, 27), but compound 112983 is the simplest yet identified.

Clinically useful drugs that target topoisomerase IB, such as the camptothecin derivatives (28), act by stabilizing the covalent complex and promoting arrest of replication and transcription forks (29). These large five ring aromatic molecules impose a physical block to ligation by intercalating directly at the cleavage site (30), and are referred to as “poisons” rather than inhibitors of these enzymes. Since compound 112983 does not lead to stabilization of the covalent complex (FIG. 8C), it is not a classic topoisomerase poison, although it does exert its effect on the covalent intermediate.² The identification of this compound from a relatively small chemical library suggests that other novel scaffolds will be found that increase or decrease the level of covalent complex, either through direct or indirect mechanisms. Molecules that act by indirect allosteric mechanisms would be of great interest as potential drugs for treatment of camptothecin resistant tumors. The high-throughput assay that we have introduced here should greatly accelerate the identification of such potentially useful molecules.

### Example 2

Using the assay described above, a chemical library was screened for the ability to inhibit human topoisomerase type 1B. Table 5 sets forth compounds identified as being inhibitors of human topoisorease type 1B.

TABLE 5
Identified inhibitors of human type lB DNA topoisomerase

| | | % | |
|---|---|---|---|
| | | Inhibition | % Inhibition of |
| | NSC | In HTS | plasmid supercoil |
| Chemical Structure | Number | screen | Relaxation |
| 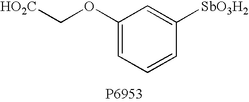 | P6953 | 100 @ 200 nM | 80% @ 100 nM |
| 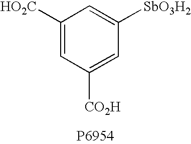 | P6954 | 100 @ 10 nM | 100 @ 10 nM |
| 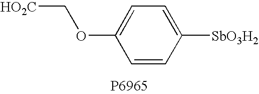 | P6965 | 100 @ 200 nM | 100 @ 1 μM |
| 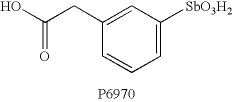 | P6970 | 100 @ 100 nM | 100 @ 50 nM |
| 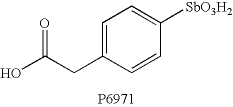 | P6971 | 100 @ 200 nM | 100 @ 1 μM |
| 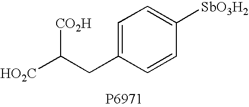 | P6982 | 50 @ 50 nM | 100 @ 50 nM |

### REFERENCES

1. Shuman, S. (1998) Vaccinia virus DNA topoisomerase: A model eukaryotic type IB enzyme, Biochim Biophys Acta 1400, 321-37.
2. O'Brien, P. J., and Herschlag, D. (1999) Catalytic promiscuity and the evolution of new enzymatic activities, Chem Biol 6, R91-R105.
3. Shuman, S. (1992) DNA strand transfer reactions catalyzed by vaccinia topoisomerase I, J Biol Chem 267, 8620-7.
4. Krogh, B. O., and Shuman, S. (2000) DNA strand transfer catalyzed by vaccinia topoisomerase: Peroxidolysis and hydroxylaminolysis of the covalent protein-DNA intermediate, Biochemistry 39, 6422-32.
5. Sekiguchi, J., and Shuman, S. (1997) Site-specific ribonuclease activity of eukaryotic DNA topoisomerase I, Mol Cell 1, 89-97.
6. Werner, R. M., and Stivers, J. T. (2000) Kinetic isotope effect studies of the reaction catalyzed by uracil DNA glycosylase: Evidence for an oxocarbonium ion-uracil anion intermediate, Biochemistry 39, 14054-14064.
7. Stivers, J. T., Harris, T. K., and Mildvan, A. S. (1997) Vaccinia DNA topoisomerase I: Evidence supporting a free rotation mechanism for DNA supercoil relaxation, Biochemistry 36, 5212-22.
8. Liu, L. F., and D'Arpa, P. (1992) Topoisomerase-targeting antitumor drugs: Mechanisms of cytotoxicity and resistance, Important Adv Oncol, 79-89.
9. Liu, L. F., Desai, S. D., Li, T. K., Mao, Y., Sun, M., and Sim, S. P. (2000) Mechanism of action of camptothecin, Ann NY Acad Sci 922, 1-10.
10. Da Fonseca, F., and Moss, B. (2003) Vacciniavirus DNA topoisomerase knockout mutant exhibits decreased infectivity associated with reduced early transcription, Proc Natl Acad Sci USA 100, 11291-6.
11. Summerer, D., and Marx, A. (2002) A molecular beacon for quantitative monitoring of the DNA polymerase reaction in real-time, Angew Chem Int Ed Engl 41, 3620-2, 3516.
12. Rizzo, J., Gifford, L. K., Zhang, X., Gewirtz, A. M., and Lu, P. (2002) Chimeric rna-DNA molecular beacon assay for ribonuclease H activity, Mol Cell Probes 16, 277-83.
13. Kwon, K., and Stivers, J. T. (2002) Fluorescence spectroscopy studies of vaccinia type IB DNA topoisomerase. Closing of the enzyme clamp is faster than DNA cleavage, J Biol Chem 277, 345-52.
14. Kuzmic, P. (1996) Program dynafit for the analysis of enzyme kinetic data: Application to hiv proteinase, Anal. Biochem. 237, 260-73.
15. Stivers, J. T., Jagadeesh, G. J., Nawrot, B., Stec, W. J., and Shuman, S. (2000) Stereochemical outcome and kinetic effects of Rp- and Sp-phosphorothioate substitutions at the cleavage site of vaccinia type i DNA topoisomerase, Biochemistry 39, 5561-72.
16. Fersht, A. (1985) Enzyme structure and mechanism, W. H. Freeman and Company, New York.
17. Segel, I. H. (1993) Enzyme kinetics, John Wiley & Sons, Inc., New York.
18. Sekiguchi, J., Stivers, J. T., Mildvan, A. S., and Shuman, S. (1996) Mechanism of inhibition of vaccinia DNA topoisomerase by novobiocin and coumermycin, J Biol Chem 271, 2313-22.
19. Sekiguchi, J., and Shuman, S. (1994) Stimulation of vaccinia topoisomerase I by nucleoside triphosphates, J Biol Chem 269, 29760-4.
20. Hwang, Y., Rowley, D., Rhodes, D., Gertsch, J., Fenical, W., and Bushman, F. (1999) Mechanism of inhibition of a vacciniavirus topoisomerase by the marine natural product sansalvamide a, Mol Pharmacol 55, 1049-53.
21. Pilch, D. S., Xu, Z., Sun, Q., LaVoie, E. J., Liu, L. F., and Breslauer, K. J. (1997) A terbenzimidazole that preferentially binds and conformationally alters structurally distinct DNA duplex domains: A potential mechanism for topoisomerase I poisoning, Proc Natl Acad Sci USA 94, 13565-70.
22. Rangarajan, M., Kim, J. S., Sim, S. P., Liu, A., Liu, L. F., and Lavoie, E. J. (2000) Topoisomerase I inhibition and cytotoxicity of 5-bromo- and 5-phenylterbenzimidazoles, Bioorg Med Chem 8, 2591-600.
23. Hwang, Y., Rhodes, D., and Bushman, F. (2000) Rapid microtiter assays for vacciniavirus topoisomerase, mammalian type ib topoisomerase and HIV-1 integrase: Application to inhibitor isolation, Nucleic Acids Res 28, 4884-92.
24. Celia, H., Hoermann, L., Schultz, P., Lebeau, L., Mallouh, V., Wigley, D. B., Wang, J. C., Mioskowski, C., and Oudet, P. (1994) Three-dimensional model of Escherichia coli gyrase B subunit crystallized in two-dimensions on novobiocin-linked phospholipid films, J Mol Biol 236, 618-28.
25. Lewis, R. J., Singh, O. M., Smith, C. V., Maxwell, A., Skarzynski, T., Wonacott, A. J., and Wigley, D. B. (1994) Crystallization of inhibitor complexes of an N-terminal 24 kDa fragment of the DNA gyrase B protein, J Mol Biol 241, 128-30.
26. Hwang, Y., Wang, B., and Bushman, F. D. (1998) Molluscum contagiosum virus topoisomerase: Purification, activities, and response to inhibitors, J Virol 72, 3401-6.
27. Yakovleva, L., Handy, C. J., Sayer, J. M., Pirrung, M., Jerina, D. M., and Shuman, S. (2004) Benzo[c]phenanthrene adducts and nogalamycin inhibit DNA transesterification by vaccinia topoisomerase, J Biol Chem.
28. Hsiang, Y. H., Liu, L. F., Wall, M. E., Wani, M. C., Nicholas, A. W., Manikumar, G., Kirschenbaum, S., Silber, R., and Potmesil, M. (1989) DNA topoisomerase I-mediated DNA cleavage and cytotoxicity of camptothecin analogues, Cancer Res 49, 4385-9.
29. Hsiang, Y. H., Lihou, M. G., and Liu, L. F. (1989) Arrest of replication forks by drug-stabilized topoisomerase I-DNA cleavable complexes as a mechanism of cell killing by camptothecin, Cancer Res 49, 5077-82.
30. Staker, B. L., Hjerrild, K., Feese, M. D., Behnke, C. A., Burgin, A. B., Jr., and Stewart, L. (2002) The mechanism of topoisomerase I poisoning by a camptothecin analog, Proc Natl Acad Sci USA 99, 15387-92.
31. Kwon, K., Nagarajan, R. and Stivers, J. T. (2004) The Ribonuclease Activity of Vaccinia DNA Topoisomerase IB: Presteady-State Kinetic and High-Throughput Inhibition Studies Using a Robust Continuous Fluorescence Assay. Biochemistry 43, 14994-15004
   Incorporation by Reference

The contents of all references, patents, pending patent applications and published patents, cited throughout this application are hereby expressly incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method for measuring the activity of a topoisomerase comprising:
contacting a topoisomerase with a duplex nucleic acid molecule that allows for multiple turnover of the topoisomerase comprising a fluorescent moiety covalently attached to one strand of the duplex nucleic acid molecule and a fluorescence quencher covalently attached to the complimentary strand of the duplex nucleic acid molecule, wherein topoisomerase activity results in measurable fluorescence from the fluorescent moiety;
measuring the fluorescence of the fluorescent moiety;
thereby measuring the activity of the topoisomerase.

2. The method of claim 1, wherein the duplex nucleic acid molecule is a deoxyribonucleic acid molecule.

3. The method of claim 2, wherein the multiple turnover of the topoisomerase is due to the duplex nucleic acid molecule further comprising one or more ribonucleotides, or analogs thereof.

4. The method of claim 3, wherein the ribonucleotide analog is a uridine ribonucleotide.

5. The method of claim 1, wherein the fluorescence is measured spectroscopically.

6. The method of claim 1, wherein the topoisomerase is a type I topoisomerase.

7. The method of claim 6, wherein the topoisomerase type I is a topoisomerase IB.

8. The method of claim 7, wherein the topoisomerase IB is a vaccinia virus topoisomerase IB.

9. The method of claim 7, wherein the duplex nucleic acid molecule is comprised of SEQ ID NO:1 and SEQ ID NO:2.

10. The method of claim 7, wherein the topoisomerase IB is a human topoisomerase IB.

11. The method of claim 10, wherein the duplex nucleic acid molecule is comprised of SEQ ID NO:3 and SEQ ID NO:4.

12. The method of claim 7, wherein the topoisomerase is a pathogen topoisomerase.

13. The method of claim 12, wherein the topoisomerase is a malaria or trypanosome topoisomerase.

14. The method of claim 6, wherein the topoisomerase type I is topoisomerase type IA.

15. The method of claim 1, wherein the topoisomerase is a type II topoisomerase.

16. The method of claim 1, further comprising contacting the topoisomerase with a candidate topoisomerase inhibitor.

17. The method of claim 15, further comprising comparing the level of activity of the topoisomerase in the absence of the candidate inhibitor to the activity in the presence of the candidate inhibitor, wherein a lower level of activity in the presence of the candidate inhibitor is indicative that the candidate inhibitor is an inhibitor.

18. A method of determining if a compound is an antiviral agent comprising:
creating an admixture comprising topoisomerase, a candidate antiviral agent, and a duplex nucleic acid molecule that allows for multiple turnover of the topoisomerase comprising a fluorescent moiety covalently attached to one strand and a fluorescence quencher covalently attached to the complimentary strand, wherein topoisomerase activity allows for measurable fluorescence emission from the fluorescent moiety;
measuring the fluorescence of the fluorescent moiety, thereby measuring the activity of the topoisomerase;
wherein, a decrease in the activity of the topoisomerase in the presence of the candidate antiviral agent compared to the level of activity of the topoisomerase in the absence of the candidate antiviral agent is indicative that the candidate inhibitor is a antiviral agent.

19. The method of claim 18, wherein the duplex nucleic acid molecule is a deoxyribonucleic acid molecule.

20. The method of claim 20, wherein the multiple turnover of the topoisomerase is due to the nucleic acid molecule further comprising one or more ribonucleotides, or analogs thereof.

21. The method of claim 20, wherein the ribonucleotide analog is a uridine ribonucleotide.

22. The method of claim 18, wherein the fluorescence is measured spectroscopically.

23. The method of claim 18, wherein the topoisomerase is a type I topoisomerase.

24. The method of claim 22, wherein the topoisomerase type I is a topoisomerase IB.

25. The method of claim 24, wherein the topoisomerase IB is a vaccinia virus topoisomerase IB.

26. The method of claim 25, wherein the duplex nucleic acid molecule is comprised of SEQ ID NO:1 and SEQ ID NO:2.

27. The method of claim 23, wherein the topoisomerase type I is topoisomerase type IA.

28. The method of claim 18, wherein the topoisomerase is a type II topoisomerase.

29. The method of claim 18, further comprising testing a library of candidate antiviral agents.

30. A method of determining if a compound is an anticancer agent comprising:
creating an admixture comprising topoisomerase, a candidate anticancer agent, and a duplex nucleic acid molecule that allows for multiple turnover of the topoisomerase comprising a fluorescent moiety covalently attached to one strand and a fluorescence quencher covalently attached to the complimentary strand, wherein topoisomerase activity allows for measurable fluorescence emission from the fluorescent moiety;
measuring the fluorescence of the fluorescent moiety, thereby measuring the activity of the topoisomerase;
wherein, a decrease in the activity of the topoisomerase in the presence of the candidate anticancer agent compared to the level of activity of the topoisomerase in the absence of the candidate anticancer agent is indicative that the candidate inhibitor is a anticancer agent.

31. The method of claim 30, wherein the duplex nucleic acid molecule is a deoxyribonucleic acid molecule.

32. The method of claim 30, wherein the multiple turnover of the topoisomerase is due to the nucleic acid molecule further comprising one or more ribonucleotides, or analogs thereof.

33. The method of claim 32, wherein the ribonucleotide analog is uridine ribonucleotide.

34. The method of claim 30, wherein the fluorescence is measured spectroscopically.

35. The method of claim 30, wherein the topoisomerase is a type I topoisomerase.

36. The method of claim 35, wherein the topoisomerase type I is a topoisomerase type IB.

37. The method of claim 36, wherein the topoisomerase IB is a human topoisomerase IB.

38. The method of claim 37, wherein the duplex nucleic acid molecule is comprised of SEQ ID NO:3 and SEQ ID NO:4.

39. The method of claim 35, wherein the topoisomerase type I is topoisomerase type IA.

40. The method of claim 30, wherein the topoisomerase is a type II topoisomerase.

41. The method of claim 30, further comprising testing a library of candidate anticancer agents.

42. A method of determining if a compound is an antitrypanosome agent comprising:
creating an admixture comprising topoisomerase, a candidate antitrypanosome agent, and a duplex nucleic acid molecule that allows for multiple turnover of the topoisomerase comprising a fluorescent moiety covalently attached to one strand and a fluorescence quencher covalently attached to the complimentary strand, wherein topoisomerase activity allows for measurable fluorescence emission from the fluorescent moiety;
measuring the fluorescence of the fluorescent moiety, thereby measuring the activity of the topoisomerase;
wherein, a decrease in the activity of the topoisomerase in the presence of the candidate anticancer agent compared to the level of activity of the topoisomerase in the absence of the candidate anticancer agent is indicative that the candidate inhibitor is a antitrypanosome agent.

43. The method of claim 42, wherein the duplex nucleic acid molecule is a deoxyribonucleic acid molecule.

44. The method of claim 42, wherein the multiple turnover of the topoisomerase is due to the nucleic acid molecule further comprising one or more ribonucleotides, or analogs thereof.

45. The method of claim 44, wherein the ribonucleotide analog is uridine ribonucleotide.

46. The method of claim 42, wherein the fluorescence is measured spectroscopically.

47. The method of claim 42, wherein the topoisomerase is a type I topoisomerase.

48. The method of claim 47, wherein the topoisomerase type I is a topoisomerase IB.

49. The method of claim 48, wherein the topoisomerase IB is a trypanosome topoisomerase IB.

50. The method of claim 47, wherein the topoisomerase type I is topoisomerase type IA.

51. The method of claim 42, wherein the topoisomerase is a type II topoisomerase.

52. The method of claim 42, further comprising testing a library of candidate anticancer agents.

53. A method of determining if a compound is an antibacterial agent comprising:
creating an admixture comprising topoisomerase, a candidate antibacterial agent, and a duplex nucleic acid molecule that allows for multiple turnover of the topoisomerase comprising a fluorescent moiety covalently attached to one strand and a fluorescence quencher covalently attached to the complimentary strand, wherein topoisomerase activity allows for measurable fluorescence emission from the fluorescent moiety;
measuring the fluorescence of the fluorescent moiety, thereby measuring the activity of the topoisomerase;
wherein, a decrease in the activity of the topoisomerase in the presence of the candidate antibacterial agent compared to the level of activity of the topoisomerase in the absence of the candidate antibacterial agent is indicative that the candidate inhibitor is a antibacterial agent.

54. The method of claim 53, wherein the duplex nucleic acid molecule is a deoxyribonucleic acid molecule.

55. The method of claim 53, wherein the multiple turnover of the topoisomerase is due to the nucleic acid molecule further comprising one or more ribonucleotides, or analogs thereof.

56. The method of claim 55, wherein the ribonucleotide analog is uridine ribonucleotide.

57. The method of claim 53, wherein the fluorescence is measured spectroscopically.

58. The method of claim 53, wherein the topoisomerase is a type I topoisomerase.

59. The method of claim 58, wherein the topoisomerase type I is a topoisomerase IB.

60. The method of claim 59, wherein the topoisomerase IB is a bacterial topoisomerase IB.

61. The method of claim 58, wherein the topoisomerase type I is topoisomerase type IA.

62. The method of claim 53, wherein the topoisomerase is a type II topoisomerase.

63. The method of claim 53, further comprising testing a library of candidate antibacterial agents.

64. A method of determining if a compound is an antimalarial agent comprising:
creating an admixture comprising topoisomerase, a candidate antimalarial agent, and a duplex nucleic acid molecule that allows for multiple turnover of the topoisomerase comprising a fluorescent moiety covalently attached to one strand and a fluorescence quencher covalently attached to the complimentary strand, wherein topoisomerase activity allows for measurable fluorescence emission from the fluorescent moiety;
measuring the fluorescence of the fluorescent moiety, thereby measuring the activity of the topoisomerase;
wherein, a decrease in the activity of the topoisomerase in the presence of the candidate antimalarial agent compared to the level of activity of the topoisomerase in the absence of the candidate antimalarial agent is indicative that the candidate inhibitor is a antimalarial agent.

65. The method of claim 64, wherein the duplex nucleic acid molecule is a deoxyribonucleic acid molecule.

66. The method of claim 64, wherein the multiple turnover of the topoisomerase is due to the nucleic acid molecule further comprising one or more ribonucleotides, or analogs thereof.

67. The method of claim 66, wherein the ribonucleotide analog is uridine ribonucleotide.

68. The method of claim 64, wherein the fluorescence is measured spectroscopically.

69. The method of claim 64, wherein the topoisomerase is a type I topoisomerase.

70. The method of claim 69, wherein the topoisomerase type I is a topoisomerase IB.

71. The method of claim 70, wherein the topoisomerase IB is a malarial topoisomerase IB.

72. The method of claim 69, wherein the topoisomerase type I is a topoisomerase type IA.

73. The method of claim 69, wherein the topoisomerase is a type II topoisomerase.

74. The method of claim 86, further comprising testing a library of candidate antimalarial agents.

75. A method for treating a subject having a topoisomerase associate disease or disorder comprising:
administering to the subject an effective amount of a compound of Formula 1: 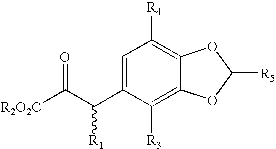
wherein: the line to R₁ indicates a single or a double bond; R₂-R₅ are each independently H, a halide, an alkyl, an aryl, a cyano, an alcohol, an amine or an amide; and R₁ is H, a halide, an alkyl, an aryl, a cyano, an alcohol, an amine or an amide.

76. The method of claim 75, wherein said topoisomerase associated disease or disorder is selected from the group consisting of cancer, viral infection or bacterial infection.

77. A method for treating a subject having a topoisomerase associate disease or disorder comprising:
administering to the subject an effective amount of a compound of Formula 2: 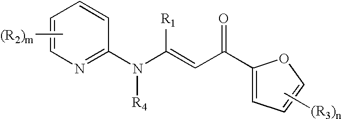
wherein: R₁ is H, a halide, an alkyl, an aryl, a cyano, an alcohol, an amine or an amide; (R₂)ₘ are each independently H, a halide, an alkyl, an aryl, a cyano, an alcohol, an amine or an amide and m=1, 2, 3 or 4; (R3)ₙ are each independently H, a halide, an alkyl, an aryl, a cyano, an alcohol, an amine or an amide and n=1, 2 or 3.

78. The method of claim 77, wherein said topoisomerase associated disease or disorder is selected from the group consisting of cancer, viral infection or bacterial infection.

79. A method for treating a subject having a topoisomerase associate disease or disorder comprising:
administering to the subject an effective amount of a compound of Formula 3: 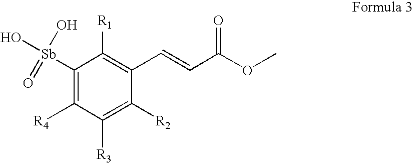
wherein: R₁-R₄ are each independently H, a halide, an alkyl, an aryl, a cyano, an alcohol, an amine or an amide.

80. The method of claim 79, wherein said topoisomerase associated disease or disorder is selected from the group consisting of cancer, viral infection or bacterial infection.

81. A method for treating a subject having a topoisomerase associate disease or disorder comprising:
administering to the subject an effective amount of a compound of Formula 4: 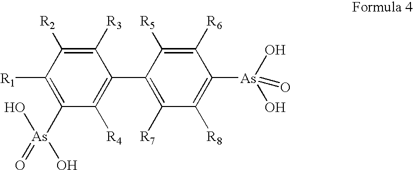
wherein: R₁-R₇ are each independently H, a halide, an alkyl, an aryl, a cyano, an alcohol, an amine or an amide.

82. The method of claim 81, wherein said topoisomerase associated disease or disorder is selected from the group consisting of cancer, viral infection or bacterial infection.

83. A method for treating a subject having a topoisomerase associate disease or disorder comprising:
administering to the subject an effective amount of a compound of Formula 5: 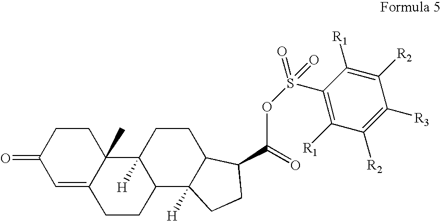
wherein: R₁-R₃ are each independently H, a halide, an alkyl, an aryl, a cyano, an alcohol, an amine or an amide.

84. The method of claim 83, wherein said topoisomerase associated disease or disorder is selected from the group consisting of cancer, viral infection or bacterial infection.

85. A method for treating a subject having a topoisomerase associate disease or disorder comprising:
administering to the subject an effective amount of a compound of Formula 6: 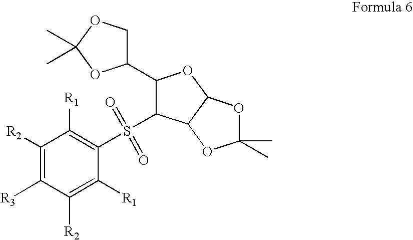
wherein: R₁-R₃ are each independently H, a halide, an alkyl, an aryl, a cyano, an alcohol, an amine or an amide.

86. The method of claim 85, wherein said topoisomerase associated disease or disorder is selected from the group consisting of cancer, viral infection or bacterial infection.

87. A method for treating a subject having a topoisomerase associate disease or disorder comprising:
administering to the subject an effective amount of a compound of Formula 7: 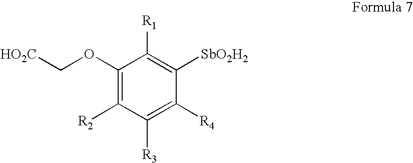
wherein: R_{1,} R_{2,} R_{3,} and R₄ are each independently H, an Alkyl, a halide, an aryl, a cyano, an alcohol, an amine or an amide.

88. The method of claim 87, wherein said topoisomerase associated disease or disorder is selected from the group consisting of cancer, viral infection or bacterial infection.

89. A method for treating a subject having a topoisomerase associate disease or disorder comprising:
administering to the subject an effective amount of a compound of Formula 8: 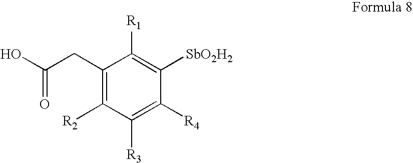
wherein: R_{1,} R_{2,} R_{3,} and R₄ are each independently H, an Alkyl, a halide, an aryl, a cyano, an alcohol, an amine or an amide.

90. The method of claim 89, wherein said topoisomerase associated disease or disorder is selected from the group consisting of cancer, viral infection or bacterial infection.

91. A method for treating a subject having a topoisomerase associate disease or disorder comprising:
administering to the subject an effective amount of a compound of Formula 9: 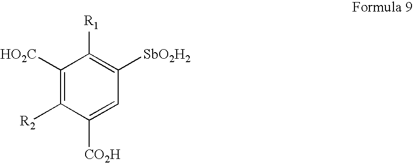
wherein: R_{1,} R_{2,} R_{3,} and R₄ are each independently H, an Alkyl, a halide, an aryl, a cyano, an alcohol, an amine or an amide.

92. The method of claim 91, wherein said topoisomerase associated disease or disorder is selected from the group consisting of cancer, viral infection or bacterial infection.

93. A method for treating a subject having a topoisomerase associate disease or disorder comprising:
administering to the subject an effective amount of a compound of Formula 10: 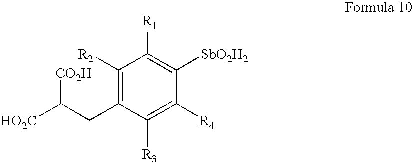
wherein: R_{1,} R_{2,} R_{3,} and R₄ are each independently H, an Alkyl, a halide, an aryl, a cyano, an alcohol, an amine or an amide.

94. The method of claim 93, wherein said topoisomerase associated disease or disorder is selected from the group consisting of cancer, viral infection or bacterial infection.

95. A method for treating a subject having a topoisomerase associate disease or disorder comprising:
administering to the subject an effective amount of a compound of Formula 11: 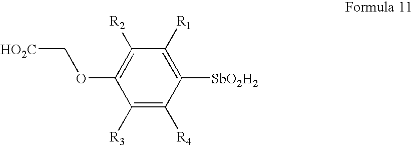
wherein: R_{1,} R_{2,} R_{3,} and R₄ are each independently H, an Alkyl, a halide, an aryl, a cyano, an alcohol, an amine or an amide.

96. The method of claim 95, wherein said topoisomerase associated disease or disorder is selected from the group consisting of cancer, viral infection or bacterial infection.

97. A method of treating a subject having a topoisomerase associated disease or disorder comprising:
administering to the subject an effective amount of a compound identified in Table 2, 3, 4 or 5;
thereby treating the subject.

98. The method of claim 97, wherein the compound is selected from the compounds listed in Table 4.

99. The method of claim 98, wherein said topoisomerase associated disease or disorder is selected from the group consisting of cancer, viral infection or bacterial infection.

100. A pharmaceutical composition for the treatment of a topoisomerase associated disease or disorder comprising a compound identified as Formula 1, Formula 2, Formula 3, Formula 4, Formula 5, Formula 6, Formula 7, Formula 8, Formula 9, Formula 10, or Formula 11 or identified in Tables 2, 3, 4, or 5 and a pharmaceutically acceptable carrier.

101. The pharmaceutical composition of claim 100, wherein the topoisomerase associated disease or disorder is selected from the group consisting of cancer, viral infection or bacterial infection.

102. A kit for the treatment of a topoisomerase associated disease or disorder comprising a pharmaceutical composition of claim 101 and instructions for use.

103. The kit of claim 102 wherein the wherein the topoisomerase associated disease or disorder is selected from the group consisting of cancer, viral infection or bacterial infection.

104. A kit for determining if a compound is a topoisomerase inhibitor comprising:
a duplex nucleic acid molecule that allows for multiple turnover of the topoisomerase comprising a fluorescent moiety covalently attached to one strand of the duplex nucleic acid molecule and a fluorescence quencher covalently attached to the complimentary strand of the duplex nucleic acid molecule, wherein topoisomerase activity results in measurable fluorescence from the fluorescent moiety, and instructions for use.

105. The kit of claim 104, further comprising topoisomerase.

106. The kit of claim 105, wherein the topoisomerase is selected from the group consisting of human, viral, pathogenic, or bacterial topoisomerase.
